(19) United States
(12) Patent Application Publication
(10) Pub. No.: US 2024/0376096 A1
(43) Pub. Date: Nov. 14, 2024

(54) SMALL MOLECULE CYCLIN DEPENDENT KINASE 4/6 (CDK4/6) AND IKZF2 (HELIOS) DEGRADERS AND METHODS OF USE THEREOF

(71) Applicant: DANA-FARBER CANCER INSTITUTE, INC., Boston, MA (US)

(72) Inventors: Alyssa Verano, Allston, MA (US); Eric Wang, Brookline, MA (US); Inchul You, Boston, MA (US); Nathanael Gray, Stanford, CA (US)

(73) Assignee: DANA-FARBER CANCER INSTITUTE, INC., Boston, MA (US)

(21) Appl. No.: 18/577,531

(22) PCT Filed: Jul. 15, 2022
(86) PCT No.: PCT/US2022/073782

§ 371 Date: Jan. 8, 2024

Related U.S. Application Data
(60) Provisional application No. 63/222,646, filed on Jul. 16, 2021.

(51) Int. Cl. C07D 471/04; A61K 31/4439; A61K 47/54; A61K 47/55; C07D 403/14

(52) U.S. Cl. CPC C07D 471/04; A61K 31/4439; A61K 47/545; A61K 47/55; C07D 403/14

(57) ABSTRACT

The present application provides bifunctional compounds that act as protein degradation inducing moieties for cyclin-dependent kinase 4 (CDK4), cyclin-dependent kinase 6 (CDK6) and Ikaros Family Zinc Finger 2 (IKZF2), also known as Helios. Also disclosed are methods for the treatment of disorders modulated by CDK4, CDK6, and IKZF2.

### RELATED APPLICATIONS

This application claims the benefit of priority under 35 U.S.C. § 119(e) to U.S. Provisional Application No. 63/222,646, filed Jul. 16, 2021, which is incorporated herein by reference in its entirety.

### GOVERNMENT SUPPORT

This disclosure was made with government support under grant number RO1 CA218278 awarded by The National Institutes of Health. The government has certain rights in the disclosure.

### BACKGROUND

Helios (IKZF2), a member of the IKZF zinc finger transcription factor family (IKZF) family, is a critical regulator of T cell activity and function. Genetic deletion of Helios resulted in an enhanced anti-tumor immune response (Kim et al., Science 350:334-339 (2015)). Notably, Helios is highly expressed in regulatory T cells (Elkord et al., Expert Opin. Biol. Ther. 12:1423-1425 (2012)), a subpopulation of T cells that restricts the activity of effector T cells. Selective deletion of Helios in regulatory T cells (Tregs) resulted in both loss of suppressive activity and acquisition of effector T cell functions (Najagawa et al., Proc. Natl. Acad. Sci. USA 113:6248-6253 (2016); Yates et al., Proc. Natl. Acad. Sci. USA 115:2162-2167 (2018)). Thus, Helios is a critical factor in restricting T cell effector function in Tregs.

Helios expression has also been reported to be upregulated in ‘exhausted’ T cells, in the settings of both chronic viral infections (Crawford et al., Immunity 40:289-302 (2014), Doering et al., Immunity 371130-1144 (2012); Scott-Browne et al., Immunity 45:1327-1340 (2016)) and tumors (Martinez et al., Immunity 42:265-278 (2015); Mognol et al., Proc. Natl. Acad. Sci. USA 114:E2776-E2785 (2017); Pereira et al., J. Leukoc. Biol. 102:601-615 (2017); Singer et al., Cell 166:1500-1511 (2016); Schietinger et al., Immunity 45:389-401 (2016)), as well as in dysfunctional chimeric antigen receptor (CAR) T cells (Long et al., Nat. Med. 21:581-590 (2015)). Overexpression or aberrant expression of Helios and various splice isoforms have been reported in several hematological malignancies, including T cell leukemias and lymphomas (Nakase et al., Exp. Hematol. 30:313-317 (2002); Tabayashi et al., Cancer Sci. 98:182-188 (2007); Asanuma et al., Cancer Sci. 104:1097-1106 (2013)). Moreover, knockdown of Helios in a model of mixed lineage leukemia (MLL)-driven myeloid leukemia potently suppressed proliferation and increased cell death (Park et al., J. Clin. Invest. 125:1286-1298 (2015); Park et al., Cell Stem Cell 24:153-165 (2019)).

Cyclin-dependent kinases (CDKs) integrate multiple signaling pathways to control either cell cycle or gene transcription. CDK1, 2, 4 and 6 are the critical enzymes that drive cell cycle transition. For example, CDK1 is a key determinant of mitotic progression, CDK2 regulates DNA replication in S phase, and CDK4/6 drives the cell cycle from G0 or G1 to S phase by phosphorylation on Rb protein to activate expression of genes involved in cell cycle control. CDK4/6 inhibitors have been shown to have potent immunostimulatory effects on T cells, in both ex vivo human T cells and in multiple in vivo murine tumor models. CDK7, 9 and 12 regulate the transcription instead of directly promoting cell cycles. CDK7 is the enzymatic component of TFIIH complex which is responsible for regulating transcription initiation, and CDK9 and CDK12 regulate transcription elongation and processing.

Deregulation of CDKs has been shown to have a significant impact on the cell state and is frequently identified as oncogenic. Numerous selective or pan-CDK small molecule inhibitors have been identified, however, most of the known inhibitors have failed in clinical trials due to the lack of high systemic drug concentration. More recently, three CDK4/6 inhibitors, abemaciclib, palbociclib, and ribociclib, have demonstrated clinical use in combination with hormone therapy to treat hormone receptor (HR)-positive human epidermal growth factor receptor 2 (HER-2)-negative metastatic breast cancer.

### SUMMARY

A first aspect of the present disclosure is directed to a bifunctional compound having a structure represented by formula (I):

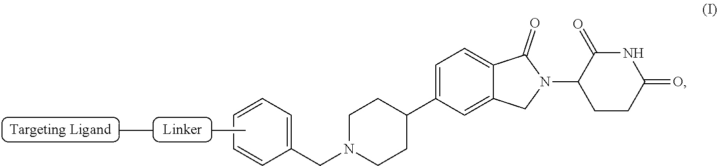

or a pharmaceutically acceptable salt or stereoisomer thereof, wherein the targeting ligand (“Targeting Ligand”) binds CDK4 and/or CDK6, the linker (“Linker”) comprises an alkylene chain or a polyethylene glycol chain.

Another aspect of the disclosure is directed to a pharmaceutical composition, comprising a therapeutically effective amount of a bifunctional compound of formula (I) or pharmaceutically acceptable salt or stereoisomer thereof, and a pharmaceutically acceptable carrier. In some embodiments, the pharmaceutical composition is in the form of a solid. In some embodiments, the pharmaceutical composition is in the form of a tablet or capsule. In some embodiments, the pharmaceutical composition is in the form of a liquid.

Another aspect of the present disclosure is directed to a method of making the bifunctional compounds of formula (I) and their pharmaceutically acceptable salts and stereoisomers.

A further aspect of the disclosure is directed to a method of treating a disease or disorder that is characterized by aberrant activity of CDK4 and/or CDK6, and Helios, comprising administering to a subject in need thereof a therapeutically effective amount of a bifunctional compound of formula (I) or pharmaceutically acceptable salt or stereoisomer thereof. In some embodiments, the disease or disorder is cancer. In some embodiments, the cancer is characterized by a solid tumor. In some embodiments, the cancer is selected from breast cancer, brain cancer, endometrial cancer, head and neck cancer, gastrointestinal cancer, lung cancer, ovarian cancer, prostate cancer, uterine cancer, hepatocellular carcinoma, liposarcoma, and melanoma. In some embodiments, the cancer is a hematological cancer. In some embodiments, the hematological cancer is selected from leukemia, lymphoma, and myeloma.

A yet further aspect of the disclosure is directed to a method of reducing the levels of CDK4 and/or CDK6, and Helios in a cell, either in vitro or in vivo, comprising contacting the cell with an effective amount of a bifunctional compound of formula (I) or pharmaceutically acceptable salt or stereoisomer thereof or pharmaceutically acceptable salt or stereoisomer thereof.

CDK4/6 inhibitors have been shown to have potent immunostimulatory effects on T cells, in both ex vivo human T cells and in multiple in vivo murine tumor models. Prior CDK4/6 inhibitors have not been shown to induce the degradation of Helios. Prior small molecule degraders of Helios have been shown to destabilize the anergic and suppressive phenotypes of regulatory T cells. The bifunctional compounds of the present disclosure may reprogram the CRL4(CRBN) E3 ligase complex to target Helios, CDK4, and/or CDK6 for ubiquitination and subsequent proteasomal degradation and thus promote anti-tumor immunity. They may also be used in combination with existing immunotherapies, such as immune checkpoint inhibitors (e.g., anti-PD-1, anti PD-L1) or cellular therapies (e.g., CAR-T cells).

Although not intending to be bound by any particular theory of operation, it is believed that by causing degradation of Helios, bifunctional compounds of the present disclosure may enhance an anti-tumor immune response by converting regulatory T cells into effector T cells, and by rescuing effector T cell function in exhausted T cells or CAR-T cells. Further it is believed that the compounds of the present disclosure exert their therapeutic (e.g., anti-cancer) effects or benefits by a combination of anti-proliferative and immunomodulatory effects. Mechanistically, the disclosed compounds are believed to induce degradation of Helios by way of forming a “molecular glue” with cereblon (CRBN).

Molecular glue compounds induce protein-protein interactions that, in the context of a ubiquitin ligase, lead to protein degradation (Stanton et al., Science 359:eaao5902 (2018)). Unlike proteolysis-targeting chimeric molecules (PROTACs), molecular glue compounds are small molecules (also known as small molecule degraders) that induce an interaction between a substrate receptor of an E3 ubiquitin ligase and a target protein leading to proteolysis of the target. Examples of molecular glues that induce proteolysis of targets include IMiDs (immune modulatory drugs; e.g., thalidomide), which generate a novel interaction between a substrate (e.g., IKZF1/3) and cereblon, a substrate receptor (also known as DCAF) for Cullin-RING ubiquitin ligase 4 (CRL4) den Besten and Lipford, Nat. Chem. Biol. 16(11):1157-1158 (2020). Unlike traditional enzyme inhibitors, these molecular glue degraders act substoichiometrically to catalyze the rapid depletion of previously inaccessible targets (Chopra et al., Drug Discov. Today. Technol. 31:5-13 (2019)). Although highly desirable, molecular glue degraders have only been found serendipitously. Strategies available for identifying or designing these compounds are limited (Slabicki et al., Nature DOI: 10.1038/s41586-020-2374-x (2020)).

In the context of the present disclosure, the compounds of formula (I) act as a molecular glue in the sense that they recruit a ubiquitin ligase, which in this case is CRBN, to the target protein, which in this case is Helios, to function as a catalyst for targeted protein degradation. In so doing, the compounds are believed to alter the substrate binding site of CRBN such that the target protein becomes a neosubstrate (Burslem et al., Chem. Rev. 117:11269-11301 (2017)). Dissociation of the molecular glue after the ubiquitination step enables subsequent function on a different molecule of the target protein (Che et al., Bioorg. Med. Chem. Lett. 28:2585-2592 (2018)).

As shown in working examples herein, the bifunctional compounds of formula (I) (also referred to herein as degraders) promote the co-degradation of CDK4, CDK6, and Helios while substantially sparing other CDKs and IKZF isoforms. Accordingly, the bifunctional compounds of the present disclosure may serve as a set of new chemical tools for CDK4, CDK6, and Helios knockdown, exemplify a broadly applicable approach to arrive at degraders that are selective over non-selective binding ligands, and may provide effective treatments for CDK4, CDK6, and Helios-mediated diseases and disorders such as cancer, neurodegenerative diseases, and autoimmune diseases.

### BRIEF DESCRIPTION OF THE DRAWINGS

FIG. 1A is a set of immunoblots of Jurkat cells treated with 1 μM of the indicated (DKY709 and Ia-1 to Ia-8) compounds for 4 hours, showing the levels of Ikaros, Helios, CDK4, CDK6, and tubulin for each compound.

FIG. 1B is a set of immunoblots of Jurkat cells treated with the Helios degrader DKY709, the triple degrader Ia-8, and the paired negative control compound 17 for 4 hours, showing the levels of Ikaros, Helios, CDK4, CDK6, and tubulin for each compound at 0.1 μM and 1 μM.

FIG. 2 is a set of immunoblots of Jurkat cells treated with the indicated compounds (palbociclib, DKY709, Ia-8, and 17) at 0.1 μM and 1 μM for 16 hours.

FIG. 3A is a set of histograms and the quantification of the DNA content using propidium iodide (PI) staining after treatment of Jurkat cells with the indicated compounds (palbociclib, DKY709, 18, Ia-6, 16, Ia-8, and 17) at 100 nM for 24 h.

FIG. 3B is a graph of normalized luminescence versus concentration (log[inhibitor], M) of the indicated compounds (palbociclib, Ia-8, and 17) assessed by CellTiterGlo after treatment of Jurkat cells for 3 d.

FIG. 4 is a graph of [IL2] for the indicated compounds in Jurkat cells upon TCR-stimulation with a-CD3/CD28.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

With respect to compounds of the present disclosure, and to the extent the following terms are used herein to further describe them, the following definitions apply.

As used herein, the term “alkyl” refers to a saturated linear or branched-chain monovalent hydrocarbon radical. To the extent not defined otherwise for any particular group in the compounds of formula (I), in one embodiment, the alkyl radical is a C₁-C₁₈ group. In other embodiments, the alkyl radical is a C₀-C₆, C₀-C₅, C₀-C₃, C₁-C₁₂, C₁-C₅, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. In some embodiments, the alkylene chain or divalent alkylene chain may be interrupted by, and/or terminate (at either or both termini) with at least one other group. In some embodiments, the alkylene chain or divalent alkylene chain may be interrupted by, and/or terminate (at either or both termini) with at least one of —O—, —S—, —N(R′)—, —C—C—, —C(O)—, —C(O)O—, —OC(O)—, —OC(O)O—, —C(NOR″)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different. To the extent not defined otherwise for any particular group in the compounds of formula (I), the alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. To the extent not defined otherwise for any particular group in the compounds of formula (I), an alkenyl includes radicals having “cis” and “trans” orientations, or alternatively, “E” and “Z” orientations. In one example, the alkenyl radical is a C₂-Cis group. In other embodiments, the alkenyl radical is a C₂-C₁₂, C₂-C₁₀, C₂-C₅, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto, which is the point of attachment. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O— alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. To the extent not defined otherwise for any particular group in the compounds of formula (I), the alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. To the extent not defined otherwise for any particular group in the compounds of formula (I), cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). To the extent not defined otherwise for any particular group in the compounds of formula (I), the term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₅, C₃-C₁₀ or C₅-C₁₀. In another embodiment, carbocyclyl, as a monocycle, includes C₃-C₅, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R′-carbocyclyl where R is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. To the extent not defined otherwise for any particular group in the compounds of formula (I), in some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur, or oxygen. In some embodiments, heterocyclyl includes 4- to 6-membered monocycles having one or more heteroatoms selected from nitrogen, sulfur, or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term “heterocyclic” embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R′ is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. To the extent not defined otherwise for any particular group in the compounds of formula (I), in some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered so as to embrace any and all such resonance structures.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not further defined for any particular group(s), representative examples of substituents may thus include alkyl, substituted alkyl (e.g., C₁-C₆, C₁₋₅, C₁₋₄, C₁₋₃, C₁₋₂, C₁), alkoxy (e.g., C₁-C₆, C₁₋₅, C₁₋₄, C₁₋₃, C₁₋₂, C₁), substituted alkoxy (e.g., C₁-C₆, C₁₋₅, C₁₋₄, C₁₋₃, C₁₋₂, C₁), haloalkyl (e.g., CF₃), alkenyl (e.g., C₂-C₆, C₂₋₅, C₂₋₄, C₂₋₃, C₂), substituted alkenyl (e.g., C₂-C₆, C₂₋₅, C₂₋₄, C₂₋₃, C₂), alkynyl (e.g., C₂-C₆, C₂₋₅, C₂₋₄, C₂₋₃, C₂), substituted alkynyl (e.g., C₂-C₆, C₂₋₅, C₂₋₄, C₂₋₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The zinc finger transcription factor Helios is critical for maintaining the identity, anergic phenotype, and suppressive activity of regulatory T cells [PMID: 26472910, 27185917, 29440380], and acute pharmacological degradation of Helios can destabilize regulatory T cell ex vivo [PMID: 34035522]. Separately, given the fundamental role cyclin-dependent kinases 4/6 (CDK4/6) have in regulating cell cycle progression, inhibitors of CDK4/6 have been found to have potent antiproliferative effects [PMID: 27030077, 29935901, 26658964]. Recently, these CDK4/6 inhibitors have also been discovered to have immunomodulatory effects, including directly promoting T cell activity [PMID: 29101163], enhancing the formation of memory T cells [PMID: 33941591], regulating regulatory T cell proliferation [PMID: 28813415], and modulating tumor cell immunogenicity [PMID: 28813415, 29160310, 30388455]. Thus, targeting both Helios and CDK4/6 may have profound effects on both reducing proliferation of tumor cells and promoting the anti-tumor immune response.

A first aspect of the present disclosure is directed to a bifunctional compound having a structure represented by formula (I):

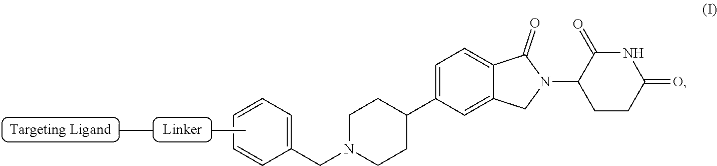

or a pharmaceutically acceptable salt or stereoisomer thereof, wherein the targeting ligand (“Targeting Ligand”) binds CDK4 and/or CDK6, the linker (“Linker”) comprises an alkylene chain or a polyethylene glycol chain.

The targeting ligand represents a moiety that binds CDK4 and/or CDK6. In some embodiments, the targeting ligand is derived from palbociclib, ribociclib, or abemaciclib. Derivatives of Palbociclib are known in the art and described, for example, in U.S. Pat. Nos. 6,936,612, 7,456,168, 10,723,730, and RE47739. Derivatives of ribociclib are known in the art and described, for example, in U.S. Pat. Nos. 8,324,225, 8,415,355, 8,685,980, 8,962,630, 9,193,732, 9,416,136, 9,868,739, and 10,799,506. Derivatives of abemaciclib are known in the art and described, for example, in U.S. Pat. No. 7,855,221.

In some embodiments, the targeting ligand is represented by any one of the following structures:

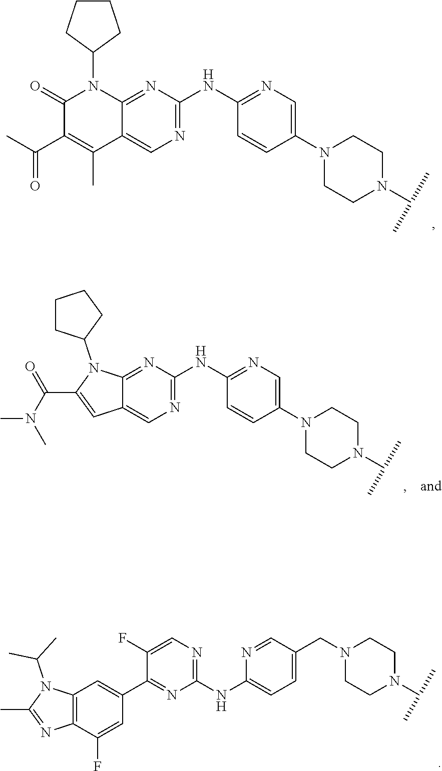

Thus, in some embodiments, the bifunctional compound has a structure represented by formula (Ia), formula (Ib), or formula (Ic):

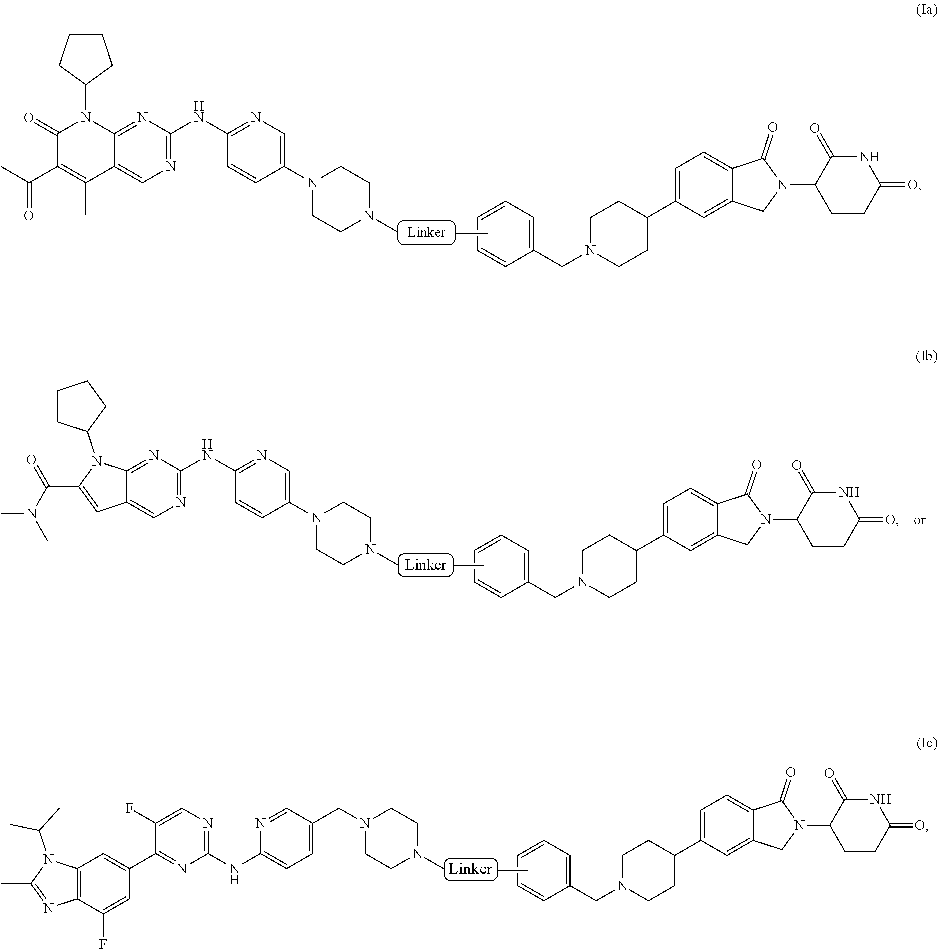

or a pharmaceutically acceptable salt or stereoisomer thereof.

In some embodiments, the linker contains an alkylene chain (e.g., having 1-20 alkylene units). In some embodiments, the linker comprises 1 to 6 alkylene units. In some embodiments, the alkylene chain or divalent alkylene chain may be interrupted by, and/or terminate (at either or both termini) with at least one of —O—, —S—, —N(R′)—, —C—C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the alkylene chain is interrupted by, and/or terminates at either or both termini with, at least one of —O— or —NH—.

In other embodiments, the linker comprises a polyethylene glycol chain. The polyethylene glycol chain may terminate (at either or both termini) with at least one of —S—, —N(R′)—, —C—C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-12 carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the polyethylene glycol chain terminates at either or both termini with at least one of —O— or —NH—. In some embodiments, the linker comprises 1 to 3 ethylene glycol units. In some embodiments, the linker is any one of the structures:

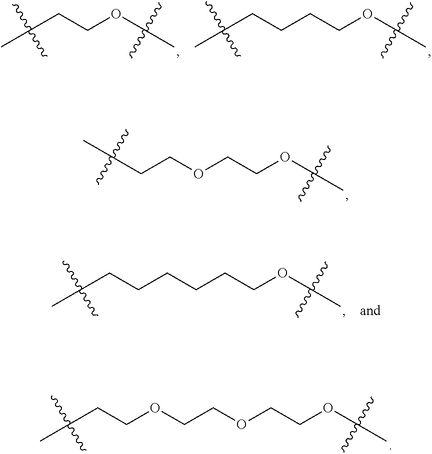 [SMILES: *CCO* ; *CCCCO* ; *CCOCCO* ; *CCCCCCO* ; *CCOCCOCCO*]

In some embodiments, the linker is attached to the phenyl group at the 3-position (meta). In some embodiments, the linker is attached to the phenyl group at the 4-position (para).

The term “binding” as it relates to interaction between the targeting ligand and two of the targeted proteins, which in this disclosure are CDK4 and/or CDK6, typically refers to an inter-molecular interaction that is preferential (also referred to herein as “selective”) in that binding of the targeting ligand with other proteins present in the cell, including other CDK isoforms, is substantially less and functionally insignificant, at least from the standpoint of degradation. The terms “selective” and “selectivity” refer to the ability of the bifunctional compound to discriminate between molecular targets. A selective dual CDK4/6 degrader described herein may have a DC₅₀ (half maximal degradation concentration) for CDK4/6 activity that is at least about 1, 2, 3, 4, 5, 6, 7, 8, 9, or 10-fold lower than the DC₅₀ for one or more of CDK1, CDK2, CDK7, CDK8, CDK9, CDK11, CDK12, CDK13, CDK14, etc., and other kinases. Thus, even though various bifunctional compounds of the present disclosure bind to other CDK proteins, albeit with similar or much less affinity, they show selective degradation of CDK4/6.

The term “binding” as it relates to interaction between the group attached to the linker opposite the Targeting Ligand in Formula (I) and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

The term “binding” as it relates to interaction between the group attached to the linker opposite the Targeting Ligand in Formula (I) and Helios, typically refers to the compounds acting as molecular glue in the sense that they recruit a ubiquitin ligase, which in this case is CRBN, to the target protein, which in this case is Helios, to function as a catalyst for targeted protein degradation.

Representative the bifunctional compounds of the present disclosure are as follows:

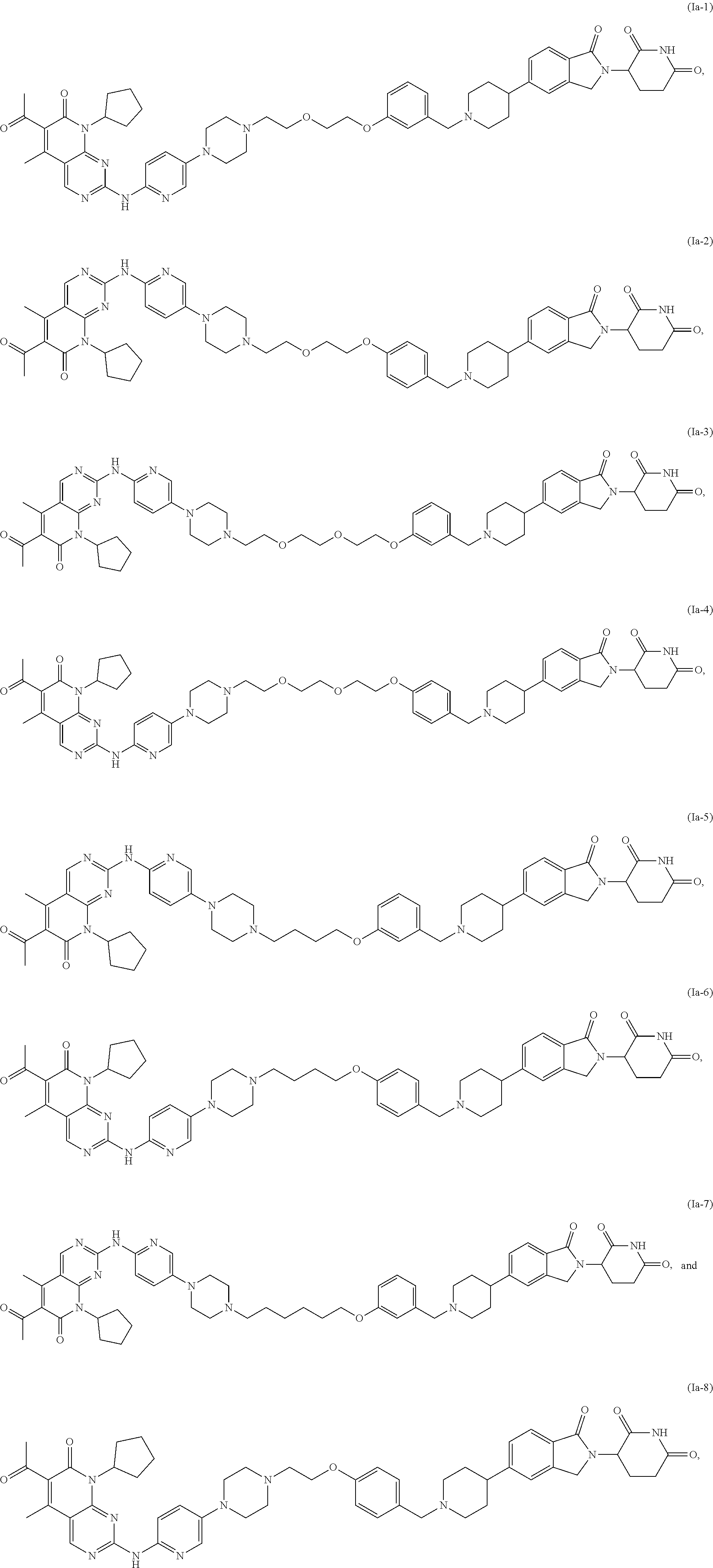

or a pharmaceutically acceptable salt or stereoisomer thereof.

Bifunctional compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present disclosure with a suitable acid or a base. Examples of pharmaceutically acceptable salts of the compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin.

Bifunctional compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present disclosure may be made and used in the form of individual isomers and substantially free of other isomers, or in the form of a mixture of various isomers, e.g., racemic mixtures of stereoisomers.

In some embodiments, the bifunctional compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e., ²H, may afford certain therapeutic advantages resulting from greater metabolic stability, for example, increased in vivo half-life or reduced dosage requirements, and thus may be advantageous in some circumstances.

In addition, bifunctional compounds of formula (I) embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In some embodiments, the present disclosure is directed to a method for making a bifunctional compound of formula (I) or a pharmaceutically acceptable salt or stereoisomer thereof. Broadly, the compounds or pharmaceutically acceptable salts or stereoisomers thereof, may be prepared by any process known to be applicable to the preparation of chemically related compounds. The compounds of the present disclosure will be better understood in connection with the synthetic schemes that described in various working examples and which illustrate non-limiting methods by which the compounds of the disclosure may be prepared.

### Pharmaceutical Compositions

Another aspect of the present disclosure is directed to a pharmaceutical composition that includes a therapeutically effective amount of a bifunctional compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bifunctional compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the present disclosure may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate, or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the present disclosure may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups, and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bifunctional compounds may be formulated for buccal or sublingual administration, examples of which include tablets, lozenges, and gels.

The bifunctional compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula (I) may be formulated for topical administration which as used herein, refers to administration intradermally by application of the formulation to the epidermis. These types of compositions are typically in the form of ointments, pastes, creams, lotions, gels, solutions, and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of formula (I) or a pharmaceutically acceptable salt or a stereoisomer thereof, or a composition including a bifunctional compound of formula (I) or a pharmaceutically acceptable salt or a stereoisomer thereof, effective in producing the desired therapeutic response in a particular patient suffering from a disease or disorder characterized or mediated by aberrant activity of any one or more of CDK4/6 and Helios. The term “therapeutically effective amount” thus includes the amount of a bifunctional compound of the disclosure or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., neuroblastoma) cells, or reduces the amount of CDK4/6 and Helios in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

Bifunctional compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day.

Individual dosages may be formulated to contain the desired dosage amount depending upon the number of times the compound is administered per day. By way of example, capsules may be formulated with from about 1 to about 200 mg of a bifunctional compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present disclosure is directed to methods of treating diseases or disorders involving aberrant (e.g., dysfunctional or dysregulated) CDK4/6 and/or Helios activity, that entails administration of a therapeutically effective amount of a bifunctional compound of formula (I) or a pharmaceutically acceptable salt or stereoisomer thereof, to a subject in need thereof. In some embodiments, the present disclosure is directed to a method of reducing the levels of CDK4 and/or CDK6, and Helios in a cell, either in vitro or in vivo, comprising contacting the cell with a bifunctional compound of formula (I).

The diseases or disorders are characterized or mediated by aberrant activity of CDK4/6 and/or Helios (e.g., elevated levels of CDK4/6 and/or Helios or otherwise functionally abnormal CDK4/6 and/or Helios relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of formula (I) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous (e.g., cell proliferative) diseases or disorders that may be amenable to treatment with the compounds of the present disclosure include inflammatory diseases and conditions, autoimmune diseases, neurodegenerative diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In some embodiments, the bifunctional compounds may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bifunctional compounds may be useful in the treatment of autoimmune diseases and disorders. As used herein, the term “autoimmune disease” refers to conditions where the immune system produces antibodies that attack normal body tissues. Representative examples of such diseases include Sjogren's syndrome, Hashirnoto thyroiditis, rheumatoid arthritis, juvenile (type 1) diabetes, polymyositis, scleroderma, Addison disease, lupus including systemic lupus erythermatosus, vitiligo, pernicious anemia, glomerulonephritis, pulmonary fibrosis, celiac disease, polymyalgia rheumatica, multiple sclerosis, ankylosing spondylitis, alopecia areata, vasculitis, and temporal arteritis.

In other embodiments, the methods are directed to treating subjects having cancer. Broadly, the bifunctional compounds of the present disclosure may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers include adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the bifunctional compounds of the present disclosure include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present disclosure entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, ! meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma,” bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present disclosure may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the disease or disorder is cancer. In some embodiments, the cancer is characterized by a solid tumor. In some embodiments, the cancer is selected from breast cancer, brain cancer, endometrial cancer, head and neck cancer, gastrointestinal cancer, lung cancer, ovarian cancer, prostate cancer, uterine cancer, hepatocellular carcinoma, liposarcoma, and melanoma. In some embodiments, the cancer is a hematological cancer. In some embodiments, the hematological cancer is selected from leukemia, lymphoma, and myeloma.

The bifunctional compounds of formula (I) may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the treatment. Therapy may also be given as an adjuvant treatment, i.e., to prevent reoccurrence of cancer in patients with no currently detectable disease or after surgical removal of a tumor. Thus, in some embodiments, the bifunctional compounds may be administered to a patient who has received another therapy, such as chemotherapy, radioimmunotherapy, surgical therapy, immunotherapy, radiation therapy, targeted therapy, or any combination thereof.

The methods of the present disclosure may entail administration of bifunctional compounds of formula (I) or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

### Combination Therapy

Bifunctional compounds of formula (I) may be used in combination or concurrently with at least one other active agent, e.g., anti-cancer agent or regimen, in treating diseases and disorders. In some embodiments, bifunctional compounds of formula (I) may be used in combination with existing immunotherapies, such as immune checkpoint inhibitors (e.g., anti-PD-1 or anti-PD-L1) or cellular therapies (e.g., CAR-T cells). The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is, in some cases, still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound of formula (I) in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006. For example, anti-cancer agents that may be suitable for use in combination with the bifunctional compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a bifunctional compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, the bifunctional compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, and depending on the particular cancer being treated, the compound of the present disclosure may be used in combination with at least one other anti-cancer agent such as Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Pembrolizumab (e.g., cervical cancer, gastric cancer, hepatocellular carcinoma, Hodgkin's lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), Regorafenib (e.g., colorectal cancer, gastrointestinal stromal tumor, and hepatocellular carcinoma), Cemiplimab (e.g., cutaneous squamous cell carcinoma (CSCC)), Avelumab (e.g., Merkel cell carcinoma, urothelial carcinoma, and renal cell carcinoma), Durvalumab (e.g., bladder and lung cancer), Atezolizumab (e.g., urothelial carcinoma, non-small cell lung cancer (NSCLC), triple-negative breast cancer (TNBC), small cell lung cancer (SCLC), and heptatocellular carcinoma (HCC)), and Ipilimumab (e.g., melanoma, non-small cell lung carcinoma (NSCLC), small cell lung cancer (SCLC), bladder cancer, and prostate cancer).

### Pharmaceutical Kits

The present bifunctional compounds and/or compositions containing them may be assembled into kits or pharmaceutical systems. Kits or pharmaceutical systems according to this aspect of the disclosure include a carrier or package such as a box, carton, tube or the like, having in close confinement therein one or more containers, such as vials, tubes, ampoules, or bottles, which contain a bifunctional compound of formula (I) or a pharmaceutical composition thereof. The kits or pharmaceutical systems of the disclosure may also include printed instructions for using the bifunctional compounds and compositions.

### EXAMPLES

These and other aspects of the present disclosure will be further appreciated upon consideration of the following Examples, which are intended to illustrate certain particular embodiments of the disclosure but are not intended to limit its scope, as defined by the claims.

### General Methods

Unless otherwise noted, reagents and solvents were used as received from commercial suppliers. All reactions were monitored using a Waters® Acquity ultra performance liquid chromatography/mass spectrometry (UPLC/MS) system using Acquity UPLC© BEH C18 column (2.1×50 mm, 1.7 m particle size). UPLC method A: solvent gradient=90% A at 0 min, 5% A at 1.8 min; method B: solvent gradient=85% A at 0 min, 1% A at 1.8 min; solvent A=0.1% formic acid in H₂O; solvent B=0.1% formic acid in acetonitrile; flow rate: 0.6 mL/min. Purification of reaction products was carried out by flash chromatography using CombiFlash©Rf with Teledyne ISCO RediSep® normal-phase silica flash columns; or Waters® high performance liquid chromatography (HPLC) system using SunFire™ C18 column (19×100 mm, 5 μm particle size): solvent gradient 0% to 99% acetonitrile in H₂O (0.035% trifluoroacetic acid (TFA) as additive); flow rate: 20 mL/min, or SunFirem C18 column (30×250 mm, 5 μm particle size): solvent gradient 0% to 99% acetonitrile in H₂O (0.035% TFA as additive); flow rate: 40 mL/min. The purity of all compounds was over 95% and was analyzed with Waters® UPLC system. ¹H NMR and ¹³C NMR spectra were obtained using Bruker Avance III spectrometers (500 MHz for ¹H, and 125 MHz for ¹³C). Chemical shifts are reported relative to deuterated methanol (δ=3.31) or dimethyl sulfoxide (DMSO) (δ=2.50) for ¹H NMR. Spectra are given in ppm (δ) and as br=broad, s=singlet, d=doublet, t=triplet, q=quartet, m=multiplet and coupling constants (J) are reported in Hertz.

### Example 1: General Synthetic Route

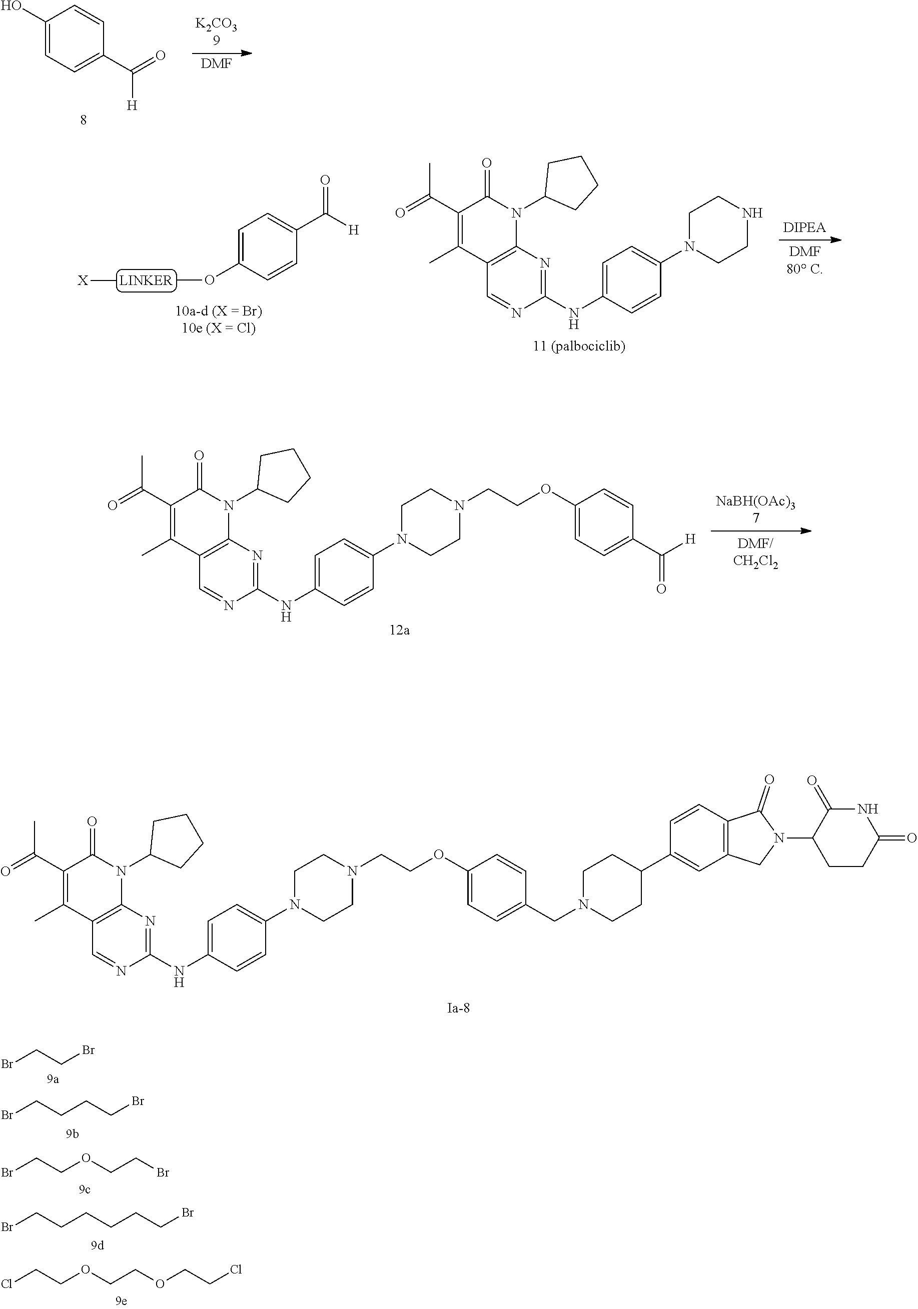

Compounds Ia-1 to Ia-8 can be prepared according to the General Synthetic Route described above, using the appropriate hydroxybenaldehyde, for example, 4-hydroxybenzaldehyde 8, or 3-hydroxybenzaldehyde, the appropriate dibromo- or dichloro-compounds, for example, 9a to 9e, and Intermediate 7, described below in Example 2. The exemplary synthesis of Ia-8 is described in Example 3.

### Example 2: Synthesis of Intermediate 7

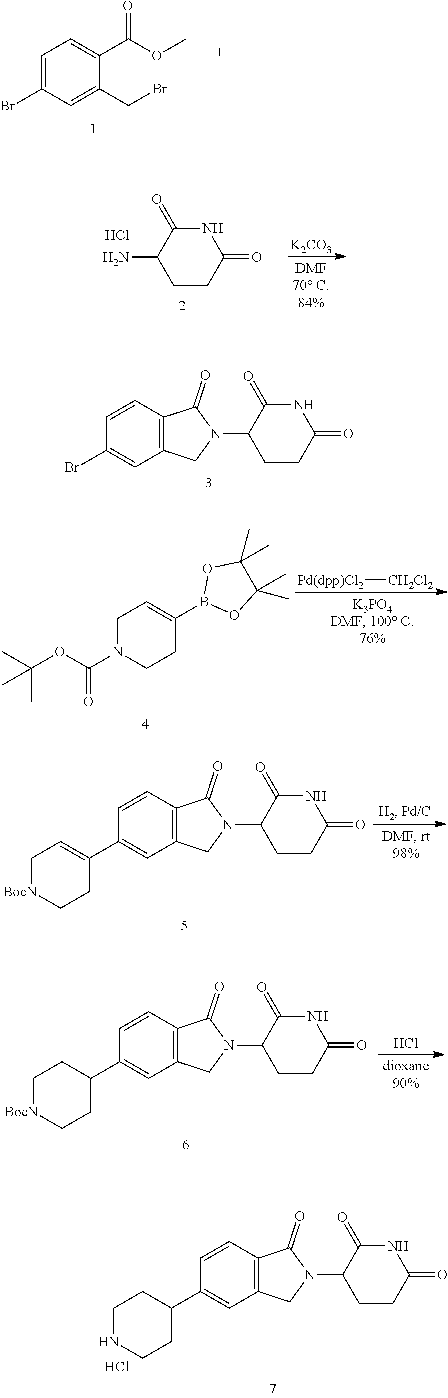

### 3-(5-bromo-1-oxoisoindolin-2-yl)piperidine-2,6-dione (3)

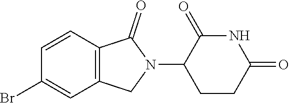 [SMILES: O=C1CCC(N2Cc3cc(Br)ccc3C2=O)C(=O)N1]

Potassium carbonate (2.29 g, 16.57 mmol) was added to a solution of methyl 4-bromo-2-(bromomethyl)benzoate 1 (1.70 g, 5.52 mmol) and 3-aminopiperidine-2,6-dione hydrochloride 2 (1 g, 6.08 mmol) in N,N-dimethylformamide (DMF, 18.4 mL, 0.30 M) and stirred at 70° C. for 17 h. The reaction mixture was concentrated in vacuo and water was added to the residue to give a precipitate, which was collected by gravity filtration and dried to yield the title compound as a solid (1.5 g, 84% yield). LC-MS (ES+): 322.9 m/z [M+H]+.

### tert-butyl 4-(2-(2,6-dioxopiperidin-3-yl)-1-oxoisoindolin-5-yl)-3,6-dihydropyridine-1(2H)-carboxylate (5)

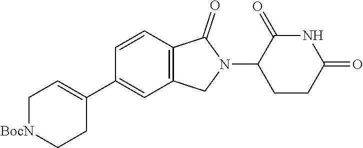 [SMILES: CC(C)(C)OC(=O)N1CC=C(c2ccc3c(c2)CN(C2CCC(=O)NC2=O)C3=O)CC1]

3-(5-bromo-1-oxoisoindolin-2-yl)piperidine-2,6-dione 3 (1.5 g, 4.64 mmol) and tert-butyl 4-(4,4,5,5-tetramethyl-1,3,2-dioxaborolan-2-yl)-3,6-dihydropyridine-1(2H)-carboxylate 4 (1.87 g, 6.03 mmol) were dissolved in DMF (30 mL, 0.155 M) under nitrogen atmosphere. Pd(dppf)Cl₂—CH₂Cl₂ (189 mg, 0.232 mmol) and potassium phosphate (K₃PO₄, 1.182 g, 5.57 mmol) were added, and the reaction mixture was stirred at 100° C. for 15 h. Upon cooling to rt, the reaction mixture was diluted with EtOAc. The organic layer was washed with water 2×, brine, dried over Na₂SO₄, filtered, and concentrated in vacuo. Purification (SiO₂: 0-100% EtOAc in hexanes) provided the title compound as a light brown solid (1.5 g, 76%). LC-MS (ES+): 426.2 m/z [M+H]+.

### tert-butyl 4-(2-(2,6-dioxopiperidin-3-yl)-1-oxoisoindolin-5-yl)piperidine-1-carboxylate (6)

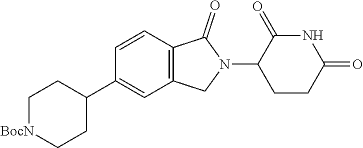 [SMILES: CC(C)(C)OC(=O)N1CCC(c2ccc3c(c2)CN(C2CCC(=O)NC2=O)C3=O)CC1]

In a 100 mL roundbottom flask equipped with a stir bar and under N₂ atmosphere, tert-butyl 4-(2-(2,6-dioxopiperidin-3-yl)-1-oxoisoindolin-5-yl)-3,6-dihydropyridine-1(2H)-carboxylate 5 (1.5 g, 3.53 mmol) was dissolved in DMF (20 mL, 0.18 M). Palladium on carbon (10 wt %, 375 mg, 0.10 equiv) was added, and the flask was equipped with a H₂ balloon and flushed with H₂. After stirring at rt for 20 h under H₂ atmosphere, the reaction mixture was filtered over celite to remove Pd catalyst and rinsed with DMF. The filtrate was concentrated in vacuo to give the title compound as an off-white solid (1.51 g, 99%), which was carried forward without purification. LC-MS 372.1 m/z [M+H-tBu]+, 328.11 m/z [M+H-Boc]+.

### 3-(1-oxo-5-(piperidin-4-yl)isoindolin-2-yl)piperidine-2,6-dione hydrochloride (7)

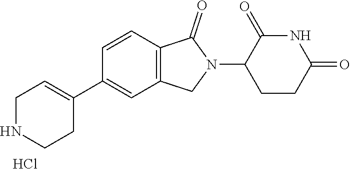 [SMILES: O=C1CCC(N2Cc3cc(C4=CCNCC4)ccc3C2=O)C(=O)N1 ; Cl]

4 M HCl in dioxane (10 mL) was added to a solution of tert-butyl 4-(2-(2,6-dioxopiperidin-3-yl)-1-oxoisoindolin-5-yl)piperidine-1-carboxylate 6 (1.51 g, 3.53 mmol) in CH₂Cl₂ (10 mL, 0.35 M) and stirred at rt for 12 h. The reaction mixture was concentrated in vacuo to give the title compound as a solid (1.156 g, 90%), which was carried forward without purification. ¹H NMR (500 MHz, DMSO-d₆) δ 10.98 (s, 1H), 8.95 (d, J=9.7 Hz, 1H), 8.88-8.72 (m, 1H), 7.70 (d, J=7.9 Hz, 1H), 7.46 (s, 1H), 7.41-7.34 (m, 1H), 5.11 (dd, J=13.3, 5.1 Hz, 1H), 4.45 (d, J=17.3 Hz, 1H), 4.32 (d, J=17.3 Hz, 1H), 3.44-3.36 (m, 2H), 3.04-2.88 (m, 4H), 2.63-2.57 (m, 1H), 2.39 (ddd, J=13.3, 4.5 Hz, 1H), 2.03-1.85 (m, 5H). LC-MS (ES+): 328.05 m/z [M+H]+.

### Example 3: Synthesis of (Ia-8)

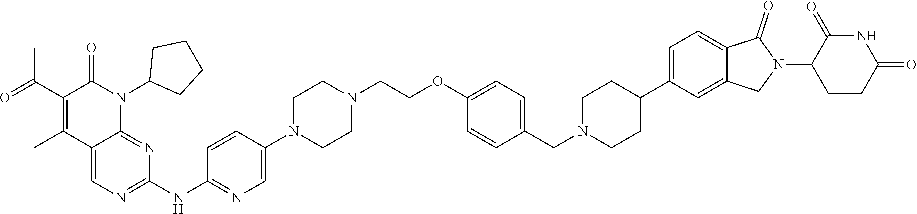 [SMILES: CC(=O)c1c(C)c2cnc(Nc3ccc(N4CCN(CCOc5ccc(CN6CCC(c7ccc8c(c7)CN(C7CCC(=O)NC7=O)C8=O)CC6)cc5)CC4)cn3)nc2n(C2CCCC2)c1=O]

### 3-(5-(1-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)benzyl)piperidin-4-yl)-1-oxoisoindolin-2-yl)piperidine-2,6-dione (Ia-8)

### 4-(2-bromoethoxy)benzaldehyde (10a)

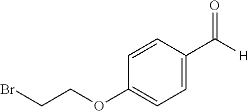 [SMILES: O=Cc1ccc(OCCBr)cc1]

Potassium carbonate (410 mg, 2.96 mmol) was added to a solution of 4-hydroxybenzaldehyde 8 (302 mg, 2.47 mmol) in DMF (10 mL, 0.25 M) and stirred at room temperature for 10 min. 1,2-dibromoethane 9a was added via syringe, and the reaction mixture was stirred at rt for 15 h. The reaction mixture was extracted with ethyl acetate (EtOAc), washed with satd aq NH₄Cl, water 2×, and brine. The organic layers were collected, dried over Na₂SO₄, filtered, and concentrated in vacuo. Purification by silica flash chromatography provided the title compound as a yellow solid (376 mg, 67% yield). LC-MS (ES+): 228.9 m/z [M+H]+.

### 4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)benzaldehyde (12a)

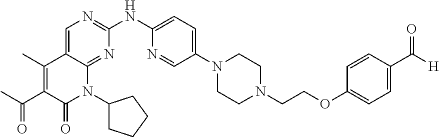 [SMILES: CC(=O)c1c(C)c2cnc(Nc3ccc(N4CCN(CCOc5ccc(C=O)cc5)CC4)cn3)nc2n(C2CCCC2)c1=O]

N,N-Diisopropylethylamine (DIPEA, 440 μL, 2.52 mmol) was added to a solution of 4-(2-bromoethoxy)benzaldehyde 10a (376 mg, 0.839 mmol) and palbociclib 11 (250 mg, 1.09 mmol) in DMF (4 mL, 0.21 M) and stirred at 80° C. for 15 h. Upon cooling to rt, the reaction mixture was extracted with EtOAc, washed with water 2×, brine, dried over Na₂SO₄, filtered, and concentrated in vacuo. Purification by silica flash chromatography (0-80% EtOAc/CH₂Cl₂, then 0-20% MeOH/CH₂Cl₂) provided the title compound as a yellow solid (310 mg, 62%). LC-MS (ES+): 596.23 m/z [M+H]+, 298.68 m/z [M+2H]2+.

### 3-(5-(1-(4-(2-(4-(6-((6-acetyl-8-cyclopentvl-5-methyl-7-oxo-7,8-dihydropyrido[2,3-d]pyrimidin-2-yl)amino)pyridin-3-yl)piperazin-1-yl)ethoxv)benzyl)piperidin-4-yl)-1-oxoisoindolin-2-yl)piperidine-2,6-dione (Ia-8)

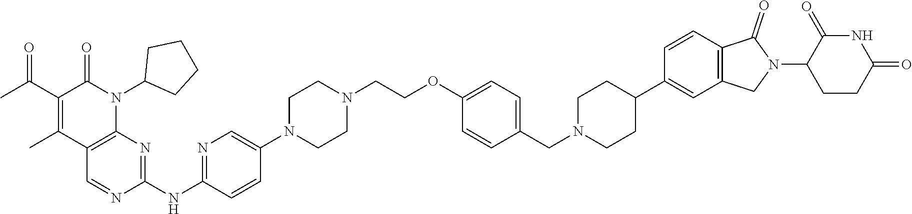 [SMILES: CC(=O)c1c(C)c2cnc(Nc3ccc(N4CCN(CCOc5ccc(CN6CCC(c7ccc8c(c7)CN(C7CCC(=O)NC7=O)C8=O)CC6)cc5)CC4)cn3)nc2n(C2CCCC2)c1=O]

Sodium triacetoxyborohydride (NaBH(OAc)₃, 64 mg, 0.300 mmol) was added to a solution of 4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)-benzaldehyde 12a (89.5 mg, 0.150 mmol) and 3-(1-oxo-5-(piperidin-4-yl)isoindolin-2-yl)piperidine-2,6-dione hydrochloride 7 (59 mg, 0.180 mmol) in 4:1 DMF/CH₂Cl₂ (3 mL, 0.05 M). Purification by silica flash chromatography (0-80% EtOAc/CH₂Cl₂, then 0-20% MeOH/CH₂Cl₂) provided the title compound as a yellow solid (5.1 mg). ¹H NMR (500 MHz, DMSO-d₆) δ 10.97 (s, 1H), 10.08 (s, 1H), 8.94 (s, 1H), 8.05 (d, J=3.0 Hz, 1H), 7.84 (d, J=9.0 Hz, 1H), 7.63 (d, J=7.9 Hz, 1H), 7.49-7.45 (m, 2H), 7.39 (d, J=8.0 Hz, 1H), 7.23 (t, J=8.4 Hz, 2H), 6.92 (d, J=8.5 Hz, 2H), 5.81 (p, J=8.5 Hz, 1H), 5.09 (dd, J=13.3, 5.1 Hz, 1H), 4.41 (d, J=17.2 Hz, 1H), 4.28 (d, J=17.4 Hz, 1H), 4.12 (t, J=5.7 Hz, 2H), 3.18-3.16 (m, 3H), 2.95-2.86 (m, 4H), 2.77 (t, J=5.6 Hz, 2H), 2.69-2.64 (m, 4H), 2.62-2.56 (m, 2H), 2.43-2.36 (m, 4H), 2.30 (s, 2H), 2.26-2.21 (m, 2H), 2.20-2.16 (m, 2H), 2.09-2.03 (m, 2H), 2.01-1.95 (m, 2H), 1.89-1.84 (m, 2H), 1.80-1.74 (m, 4H), 1.72-1.67 (m, 2H), 1.61-1.55 (m, 2H). LC-MS (ES+): 907.48 m/z [M+H]+, 454.44 m/z [M+2H]2+.

### Example 4: Synthesis of (Ia-1)

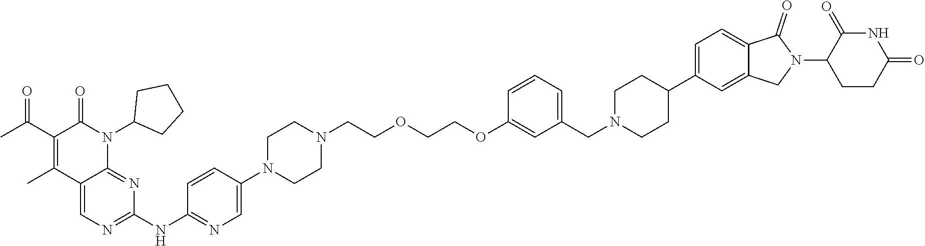 [SMILES: CC(=O)c1c(C)c2cnc(Nc3ccc(N4CCN(CCOCCOc5cccc(CN6CCC(c7ccc8c(c7)CN(C7CCC(=O)NC7=O)C8=O)CC6)c5)CC4)cn3)nc2n(C2CCCC2)c1=O]

### 3-(5-(1-(3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)benzyl)piperidin-4-yl)-1-oxoisoindolin-2-yl)piperidine-2,6-dione (Ia-1)

The title compound was prepared according to Example 1. ¹H NMR (500 MHz, DMSO-d₆) δ 10.96 (s, 1H), 10.08 (s, 1H), 8.94 (s, 1H), 8.03 (d, J=2.8 Hz, 1H), 7.84 (d, J=9.0 Hz, 1H), 7.61 (d, J=7.8 Hz, 1H), 7.46 (s, 1H), 7.44 (dd, J=9.1, 2.8 Hz, 1H), 7.37 (d, J=7.9 Hz, 1H), 7.23 (t, J=8.0 Hz, 1H), 6.94-6.87 (m, 2H), 6.83 (dd, 1H), 5.81 (p, J=8.8 Hz, 1H), 5.08 (dd, J=13.3, 5.0 Hz, 1H), 4.39 (d, J=17.2 Hz, 1H), 4.26 (d, J=17.2 Hz, 1H), 4.12-4.07 (m, 2H), 3.77-3.72 (m, 2H), 3.63 (t, J=5.6 Hz, 2H), 3.47 (s, 2H), 3.15-3.09 (m, 4H), 2.95-2.85 (m, 3H), 2.64-2.54 (m, 8H), 2.42 (s, 3H), 2.37 (dd, J=13.2, 4.5 Hz, 1H), 2.30 (s, 3H), 2.27-2.19 (m, 2H), 2.09-1.94 (m, 3H), 1.90-1.83 (m, 2H), 1.81-1.64 (m, 6H), 1.62-1.53 (m, 2H). LC-MS (ES+): 951.54 m/z [M+H]+, 476.48 m/z [M+2H]2+.

### Example 5: Synthesis of (Ia-2)

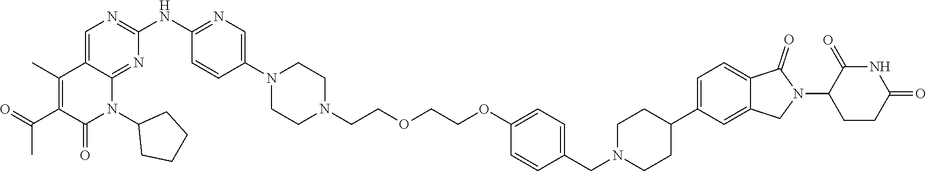 [SMILES: CC(=O)c1c(C)c2cnc(Nc3ccc(N4CCN(CCOCCOc5ccc(CN6CCC(c7ccc8c(c7)CN(C7CCC(=O)NC7=O)C8=O)CC6)cc5)CC4)cn3)nc2n(C2CCCC2)c1=O]

3-(5-(1-(4-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)benzyl)piperidin-4-yl)-1-oxoisoindolin-2-yl)piperidine-2,6-dione (Ia-2).

The title compound was prepared according to Example 1. ¹H NMR (500 MHz, DMSO-d₆) δ 10.97 (s, 1H), 10.09 (s, 1H), 8.94 (s, 1H), 8.04 (d, J=2.9 Hz, 1H), 7.84 (d, J=9.0 Hz, 1H), 7.62 (d, J=7.9 Hz, 1H), 7.48-7.42 (m, 2H), 7.37 (d, J=7.8 Hz, 1H), 7.33-7.20 (m, 2H), 6.93 (d, J=8.1 Hz, 2H), 5.81 (p, J=8.8 Hz, 1H), 5.09 (dd, J=13.3, 5.1 Hz, 1H), 4.40 (d, J=17.2 Hz, 1H), 4.27 (d, J=17.2 Hz, 1H), 4.14-4.05 (m, 2H), 3.76-3.72 (m, 2H), 3.69-3.42 (m, 4H), 3.21-2.84 (m, 7H), 2.74-2.54 (m, 8H), 2.42 (s, 3H), 2.40-2.33 (m, 1H), 2.30 (s, 3H), 2.28-2.18 (m, 3H), 2.02-1.94 (m, 1H), 1.93-1.68 (m, 9H), 1.62-1.53 (m, 2H). LC-MS (ES+): 951.56 m/z [M+H]+, 476.48 m/z [M+2H]2+.

### Example 6: Synthesis of (Ia-3)

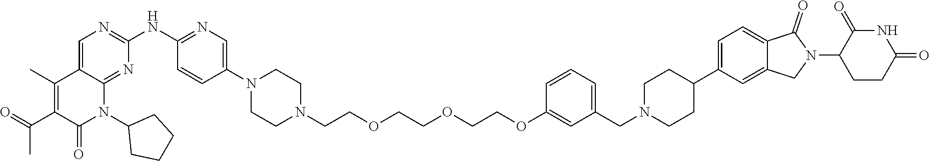 [SMILES: CC(=O)c1c(C)c2cnc(Nc3ccc(N4CCN(CCOCCOCCOc5cccc(CN6CCC(c7ccc8c(c7)CN(C7CCC(=O)NC7=O)C8=O)CC6)c5)CC4)cn3)nc2n(C2CCCC2)c1=O]

### 3-(5-(1-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)ethoxy)benzyl)piperidin-4-yl)-1-oxoisoindolin-2-yl)piperidine-2,6-dione (Ia-3)

The title compound was prepared according to Example 1. ¹H NMR (500 MHz, DMSO-d₆) δ 10.97 (s, 1H), 10.08 (s, 1H), 8.94 (s, 1H), 8.02 (d, J=2.9 Hz, 1H), 7.84 (d, J=9.0 Hz, 1H), 7.61 (d, J=7.8 Hz, 1H), 7.46 (s, 1H), 7.42 (dd, J=9.1, 3.0 Hz, 1H), 7.36 (d, J=7.9 Hz, 1H), 7.23 (t, J=8.0 Hz, 1H), 6.91-6.88 (m, 2H), 6.83 (dd, J=8.1, 2.3 Hz, 1H), 5.83-5.78 (m, 1H), 5.09 (dd, J=13.3, 5.1 Hz, 1H), 4.39 (d, J=17.2 Hz, 1H), 4.27 (d, J=17.2 Hz, 1H), 4.08 (dd, J=5.5, 3.7 Hz, 2H), 3.76 (dd, J=5.5, 3.7 Hz, 2H), 3.62-3.59 (m, 3H), 3.58-3.54 (m, 6H), 3.13-3.10 (m, 4H), 2.92-2.89 (m, 2H), 2.59-2.53 (m, 8H), 2.42 (s, 3H), 2.30 (s, 3H), 1.90 (s, 3H), 1.78-1.67 (m, 8H), 1.59-1.54 (m, 3H). LC-MS (ES+): 995.58 m/z [M+H]+, 498.47 m/z [M+2H]2+.

### Example 7: Synthesis of (Ia-4)

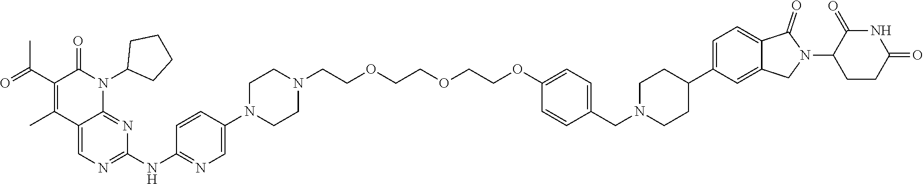 [SMILES: CC(=O)c1c(C)c2cnc(Nc3ccc(N4CCN(CCOCCOCCOc5ccc(CN6CCC(c7ccc8c(c7)CN(C7CCC(=O)NC7=O)C8=O)CC6)cc5)CC4)cn3)nc2n(C2CCCC2)c1=O]

### 3-(5-(1-(4-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)ethoxy)benzyl)piperidin-4-yl)-1-oxoisoindolin-2-yl)piperidine-2,6-dione (Ia-4)

The title compound was prepared according to Example 1. ¹H NMR (500 MHz, DMSO-d₆) δ 10.97 (s, 1H), 10.16 (s, 1H), 8.95 (s, 1H), 8.02 (s, 1H), 7.88 (d, J=8.5 Hz, 1H), 7.65 (d, J=7.8 Hz, 1H), 7.60-7.51 (m, 2H), 7.47 (d, J=7.6 Hz, 1H), 7.41 (s, 1H), 7.33 (d, J=7.6 Hz, 1H), 7.02 (d, J=8.4 Hz, 2H), 5.81 (p, J=8.8 Hz, 1H), 5.08 (dd, J=13.2, 5.0 Hz, 1H), 4.41 (d, J=17.3 Hz, 1H), 4.28 (d, J=17.3 Hz, 1H), 4.16-4.10 (m, 2H), 3.80-3.74 (m, 3H), 3.65-3.57 (m, 6H), 3.23-3.08 (m, 5H), 3.04 (q, J=7.2 Hz, 3H), 3.00-2.85 (m, 4H), 2.63-2.55 (m, 2H), 2.41 (s, 4H), 2.30 (s, 3H), 2.27-2.05 (m, 5H), 1.99-1.84 (m, 6H), 1.79-1.72 (m, 2H), 1.59-1.54 (m, 2H). LC-MS (ES+): 995.58 m/z [M+H]+, 498.48 m/z [M+2H]2+.

### Example 8: Synthesis of (Ia-5)

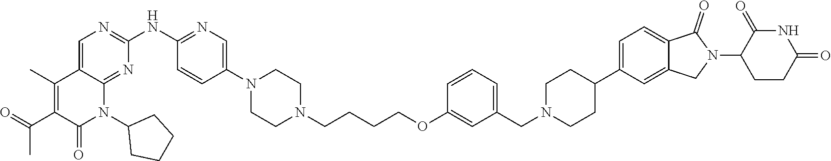 [SMILES: CC(=O)c1c(C)c2cnc(Nc3ccc(N4CCN(CCCCOc5cccc(CN6CCC(c7ccc8c(c7)CN(C7CCC(=O)NC7=O)C8=O)CC6)c5)CC4)cn3)nc2n(C2CCCC2)c1=O]

### 3-(5-(1-(3-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butoxy)benzyl)piperidin-4-yl)-1-oxoisoindolin-2-yl)piperidine-2,6-dione (Ia-5)

The title compound was prepared according to Example 1. ¹H NMR (500 MHz, DMSO-d₆) δ 10.97 (s, 1H), 10.08 (s, 1H), 8.94 (s, 1H), 8.04 (d, J=2.8 Hz, 1H), 7.84 (d, J=9.0 Hz, 1H), 7.62 (d, J=7.8 Hz, 1H), 7.48-7.43 (m, 2H), 7.38 (d, J=7.8 Hz, 1H), 7.23 (t, J=7.8 Hz, 1H), 6.93-6.87 (m, 2H), 6.82 (d, J=7.1 Hz, 1H), 5.81 (p, J=8.8 Hz, 1H), 5.09 (dd, J=13.3, 5.1 Hz, 1H), 4.40 (d, J=17.2 Hz, 1H), 4.27 (d, J=17.2 Hz, 1H), 4.05-3.96 (m, 3H), 3.17-3.12 (m, 4H), 2.99-2.84 (m, 3H), 2.61-2.52 (m, 4H), 2.44-2.35 (m, 7H), 2.30 (s, 3H), 2.27-2.17 (m, 4H), 1.98-1.94 (m, 1H), 1.90-1.83 (m, 3H), 1.79-1.71 (m, 8H), 1.65-1.56 (m, 5H). LC-MS (ES+): 935.53 m/z [M+H]+, 468.45 m/z [M+2H]2+.

### Example 9: Synthesis of (Ia-6)

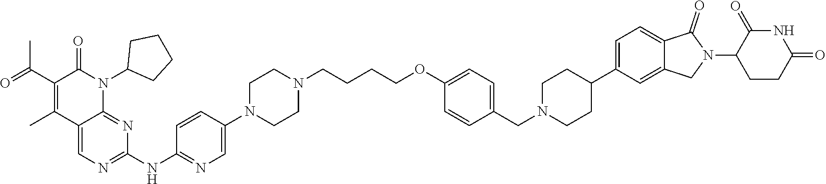 [SMILES: CC(=O)c1c(C)c2cnc(Nc3ccc(N4CCN(CCCCOc5ccc(CN6CCC(c7ccc8c(c7)CN(C7CCC(=O)NC7=O)C8=O)CC6)cc5)CC4)cn3)nc2n(C2CCCC2)c1=O]

### 3-(5-(1-(4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butoxy)benzyl)piperidin-4-yl)-1-oxoisoindolin-2-yl)piperidine-2,6-dione (Ia-6)

The title compound was prepared according to Example 1. ¹H NMR (500 MHz, DMSO-d₆) δ 10.97 (s, 1H), 10.09 (s, 1H), 8.94 (s, 1H), 8.05 (d, J=2.8 Hz, 1H), 7.84 (d, J=9.0 Hz, 1H), 7.63 (d, J=7.8 Hz, 1H), 7.50-7.43 (m, 2H), 7.38 (d, J=7.8 Hz, 1H), 7.28 (s, 2H), 6.92 (d, J=6.7 Hz, 2H), 5.81 (p, J=8.7 Hz, 1H), 5.09 (dd, J=13.3, 5.1 Hz, 1H), 4.41 (d, J=17.3 Hz, 1H), 4.28 (d, J=17.2 Hz, 1H), 4.05-3.94 (m, 3H), 3.16 (s, 4H), 3.04-2.85 (m, 3H), 2.62-2.53 (m, 4H), 2.46-2.35 (m, 7H), 2.34-2.19 (m, 7H), 2.01-1.96 (m, 1H), 1.87 (s, 3H), 1.82-1.53 (m, 13H). LC-MS (ES+): 935.54 m/z [M+H]+, 468.46 m/z [M+2H]2+.

### Example 10: Synthesis of (Ia-7)

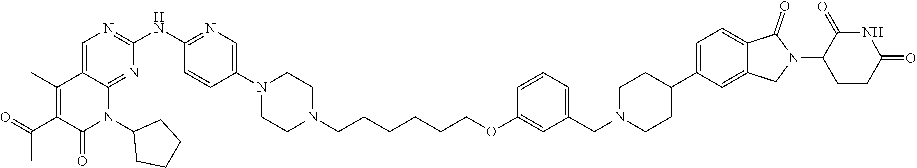 [SMILES: CC(=O)c1c(C)c2cnc(Nc3ccc(N4CCN(CCCCCCOc5cccc(CN6CCC(c7ccc8c(c7)CN(C7CCC(=O)NC7=O)C8=O)CC6)c5)CC4)cn3)nc2n(C2CCCC2)c1=O]

### 3-(5-(1-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)benzyl)piperidin-4-yl)-1-oxoisoindolin-2-yl)piperidine-2,6-dione (Ia-7)

The title compound was prepared according to Example 1. ¹H NMR (500 MHz, DMSO-d₆) δ 10.97 (s, 1H), 10.08 (s, 1H), 8.95 (s, 1H), 8.04 (d, J=2.6 Hz, 1H), 7.84 (d, J=9.0 Hz, 1H), 7.62 (d, J=7.8 Hz, 1H), 7.48-7.43 (m, 2H), 7.38 (d, J=7.9 Hz, 1H), 7.21 (d, J=8.1 Hz, 2H), 6.87 (d, J=8.3 Hz, 2H), 5.82 (p, J=8.8 Hz, 1H), 5.09 (dd, J=13.3, 5.1 Hz, 1H), 4.40 (d, J=17.2 Hz, 1H), 4.27 (d, J=17.2 Hz, 1H), 3.94 (t, J=6.3 Hz, 2H), 3.16-3.12 (m, 4H), 2.95-2.86 (m, 3H), 2.59 (d, J=16.9 Hz, 2H), 2.42 (s, 3H), 2.38 (dd, J=13.4, 4.7 Hz, 1H), 2.35-2.28 (m, 6H), 2.28-2.20 (m, 3H), 2.05-1.95 (m, 3H), 1.92-1.85 (m, 3H), 1.78-1.67 (m, 8H), 1.60-1.55 (m, 2H), 1.51-1.41 (m, 5H), 1.39-1.34 (m, 2H). LC-MS (ES+): 963.60 m/z [M+H]+, 482.50 m/z [M+2H]2+.

### Example 11: Synthesis (16)

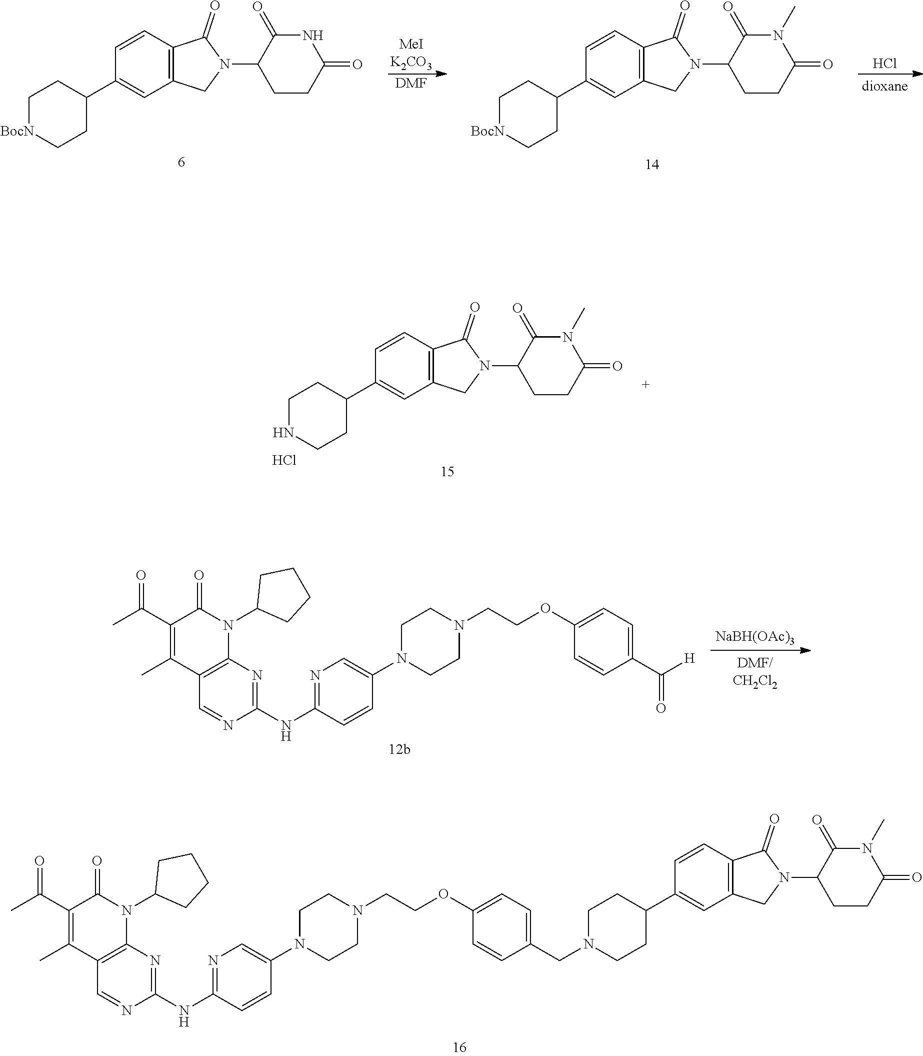

### tert-butyl 4-(2-(1-methyl-2,6-dioxopiperidin-3-yl)-1-oxoisoindolin-5-yl)piperidine-1l-carboxylate (14)

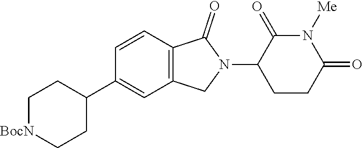 [SMILES: CN1C(=O)CCC(N2Cc3cc(C4CCN(C(=O)OC(C)(C)C)CC4)ccc3C2=O)C1=O]

Potassium carbonate was added to a solution of tert-butyl 4-(2-(2,6-dioxopiperidin-3-yl)-1-oxoisoindolin-5-yl)piperidine-1-carboxylate 6 (350 mg, 0.819 mmol) in DMF (12 mL, 0.07 M) and stirred at rt for 10 min. Methyl iodide (0.080 mL, 1.23 mmol) was added and stirred at rt for 6 h. The reaction mixture was diluted with EtOAc and washed with water 2× and brine. The organic layer was dried over Na₂SO₄, filtered, and concentrated in vacuo. Purification (SiO₂: 0-100% EtOAc in hexanes) gave the title compound (340 mg, 94%). LC-MS (ES+): 386.14 m/z [M+H−tBu]+, 342.15 m/z [M+H−Boc]+.

### 1-methyl-3-(1-oxo-5-(piperidin-4-yl)isoindolin-2-yl)piperidine-2,6-dione hydrochloride (15)

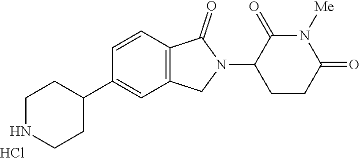 [SMILES: CN1C(=O)CCC(N2Cc3cc(C4CCNCC4)ccc3C2=O)C1=O ; Cl]

4 M HCl in dioxane (4 mL) was added to a solution of tert-butyl 4-(2-(1-methyl-2,6-dioxopiperidin-3-yl)-1-oxoisoindolin-5-yl)piperidine-1-carboxylate 14 (340 mg, 0.77 mmol) in CH₂Cl₂ (4 mL) and stirred at rt for 12 h. The reaction mixture was concentrated in vacuo to give the title compound as a solid (278 mg, 91%), which was carried forward without purification. ¹H NMR (500 MHz, DMSO-d₆) δ 9.14-9.01 (m, 2H), 7.70 (d, J=7.5 Hz, 1H), 7.46 (s, 1H), 7.37 (s, 1H), 5.17 (dd, J=12.8, 4.2 Hz, 1H), 4.44 (d, J=17.1 Hz, 1H), 4.31 (d, J=17.0 Hz, 1H), 3.36 (d, J=11.2 Hz, 2H), 3.03-2.94 (m, 7H), 2.76 (d, J=16.7 Hz, 1H), 2.43-2.34 (m, 1H), 2.02-1.90 (m, 5H). LC-MS (ES+): 342.1 m/z [M+H]+.

### 3-(5-(1-(4-(4-(4-(6-((6-acetyl-8-cyclopentvl-5-methyl-7-oxo-7,8-dihydropyrido[2,3-d]pyrimidin-2-yl)amino)pyridin-3-yl)piperazin-1-yl)butoxv)benzyl)piperidin-4-yl)-1-oxoisoindolin-2-yl)-1-methylpiperidine-2,6-dione (16)

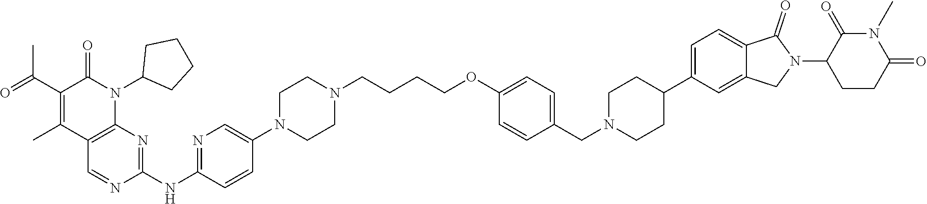 [SMILES: CC(=O)c1c(C)c2cnc(Nc3ccc(N4CCN(CCCCOc5ccc(CN6CCC(c7ccc8c(c7)CN(C7CCC(=O)N(C)C7=O)C8=O)CC6)cc5)CC4)cn3)nc2n(C2CCCC2)c1=O]

Reductive amination of 1-methyl-3-(1-oxo-5-(piperidin-4-yl)isoindolin-2-yl)piperidine-2,6-dione hydrochloride 15 and 4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butoxy)benzaldehyde 12b was carried out according to the method described in Example 3, to provide the title compound. ¹H NMR (500 MHz, DMSO-d₆) δ 10.97 (s, 1H), 10.08 (s, 1H), 8.94 (s, 1H), 8.05 (d, J=3.0 Hz, 1H), 7.84 (d, J=9.0 Hz, 1H), 7.63 (d, J=7.9 Hz, 1H), 7.49-7.45 (m, 2H), 7.39 (d, J=8.0 Hz, 1H), 7.23 (t, J=8.4 Hz, 2H), 6.92 (d, J=8.5 Hz, 2H), 5.81 (p, J=8.5 Hz, 1H), 5.09 (dd, J=13.3, 5.1 Hz, 1H), 4.41 (d, J=17.2 Hz, 1H), 4.28 (d, J=17.4 Hz, 1H), 4.12 (t, J=5.7 Hz, 2H), 3.19-3.15 (m, 4H), 2.97-2.86 (m, 5H), 2.77 (t, J=5.6 Hz, 2H), 2.69-2.56 (m, 7H), 2.42-2.36 (m, 4H), 2.30 (s, 3H), 2.26-2.21 (m, 2H), 2.18 (s, 2H), 2.09-2.03 (m, 2H), 2.01-1.95 (m, 2H), 1.89-1.84 (m, 2H), 1.81-1.53 (m, 11H). LC-MS (ES+): 949.6 m/z [M+H]+, 475.5 m/z [M+2H]2+.

### Example 12: Synthesis of (17)

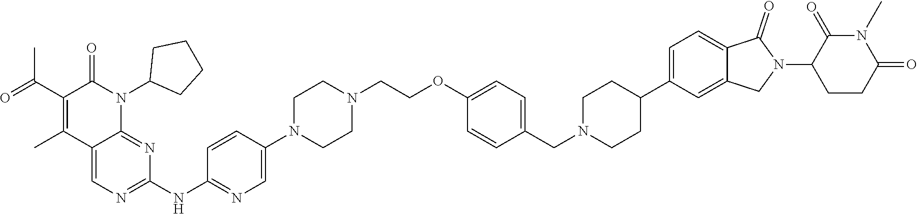 [SMILES: CC(=O)c1c(C)c2cnc(Nc3ccc(N4CCN(CCOc5ccc(CN6CCC(c7ccc8c(c7)CN(C7CCC(=O)N(C)C7=O)C8=O)CC6)cc5)CC4)cn3)nc2n(C2CCCC2)c1=O]

### 3-(5-(1-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethoxy)benzyl)piperidin-4-yl)-1-oxoisoindolin-2-yl)-1-methylpiperidine-2,6-dione (17)

Reductive amination of 1-methyl-3-(1-oxo-5-(piperidin-4-yl)isoindolin-2-yl)piperidine-2,6-dione hydrochloride 15 and 12a was carried out according to the method described in Example 3, to provide the title compound. ¹H NMR (500 MHz, DMSO-d₆) δ 10.08 (s, 1H), 8.95 (s, 1H), 8.05 (d, J=3.0 Hz, 1H), 7.84 (d, J=9.0 Hz, 1H), 7.64 (d, J=7.8 Hz, 1H), 7.50-7.45 (m, 2H), 7.39 (d, J=7.9 Hz, 1H), 7.24 (d, J=8.1 Hz, 2H), 6.92 (d, J=8.1 Hz, 2H), 5.81 (p, J=8.8 Hz, 1H), 5.16 (dd, J=13.4, 5.1 Hz, 1H), 4.41 (d, J=17.2 Hz, 1H), 4.27 (d, J=17.1 Hz, 1H), 4.12 (t, J=5.7 Hz, 2H), 3.45 (s, 2H), 3.17 (t, J=5.1 Hz, 4H), 3.00 (s, 3H), 2.95 (d, J=5.5 Hz, 2H), 2.77 (d, J=5.2 Hz, 3H), 2.73 (d, J=3.8 Hz, 1H), 2.66 (t, J=5.0 Hz, 4H), 2.42 (s, 3H), 2.40-2.35 (m, 1H), 2.30 (s, 3H), 2.24 (t, J=9.7 Hz, 2H), 2.02-1.97 (m, 2H), 1.88 (q, J=8.5, 7.4 Hz, 2H), 1.76 (d, J=9.3 Hz, 5H), 1.70 (d, J=11.8 Hz, 1H), 1.57 (q, J=6.0 Hz, 2H), 1.23 (d, J=2.8 Hz, 1H). LC-MS (ES+): 920.51 m/z [M+H]+.

### Example 13: Co-degradation of CDK4, CDK6, and Helios by Ia-1 to Ia-8

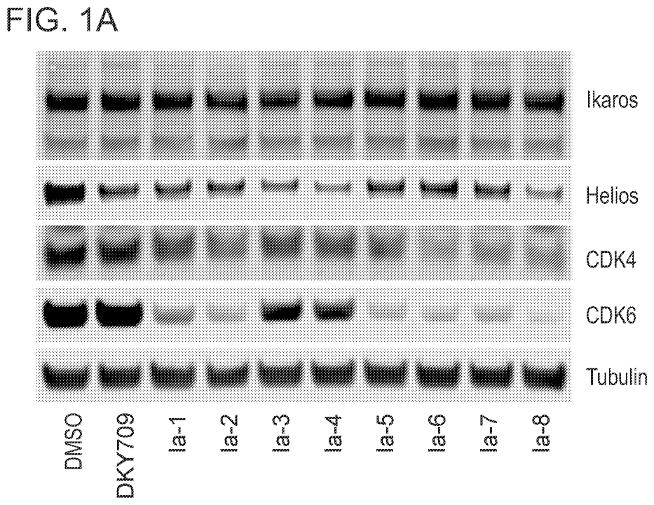

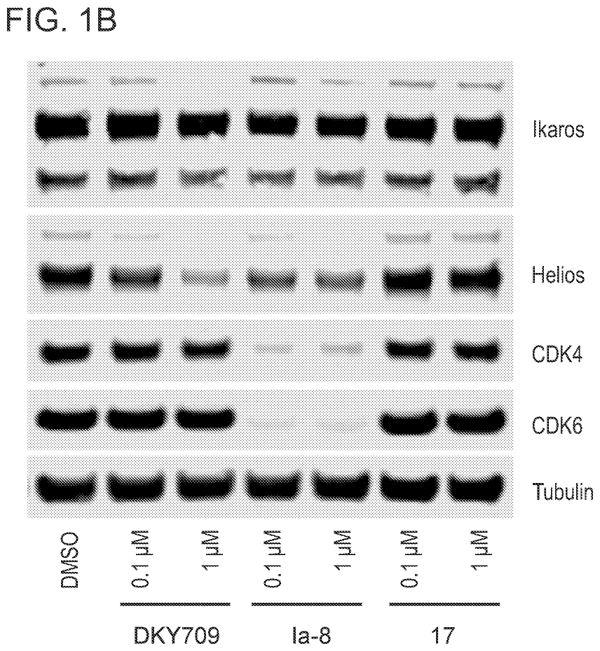

As shown in FIG. 1A, immunoblots from Jurkat cells treated with the indicated compounds (DKY709, and Ia-1 to Ia-8) for 4 h revealed co-degradation of CDK4, CDK6, and Helios while sparing Ikaros, demonstrating the feasibility of re-directing the neo-substrate specificity of the E3 ligase-binding component of a bifunctional degrader molecule. The structure of DKY709 (3-(5-(1-benzylpiperidin-4-yl)-1-oxoisoindolin-2-yl)piperidine-2,6-dione) is shown here:

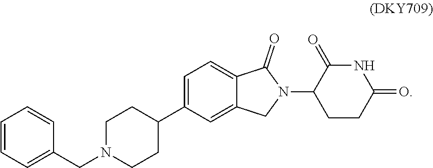 [SMILES: O=C1CCC(N2Cc3cc(C4CCN(Cc5ccccc5)CC4)ccc3C2=O)C(=O)N1]

### Example 14: Co-Degradation of CDK4, CDK6, and Helios by Ia-8

As shown in FIG. 1B, immunoblots from Jurkat cells treated with 1 μM of the indicated compounds (DKY709, the triple degrader Ia-8, and the paired negative control compound 17) for 4 h. The parental Helios degrader DKY709 only induced degradation of Helios but did not affect CDK4 or CDK6 abundance, while the triple degrader Ia-8 induced the degradation of CDK4, CDK6, and Helios, but not Ikaros in Jurkat cells. The negative control compound 17 had no degradation activity.

### Example 15: Protein Abundance of CDK4, CDK6. Helios, and Phosphorylated Rb

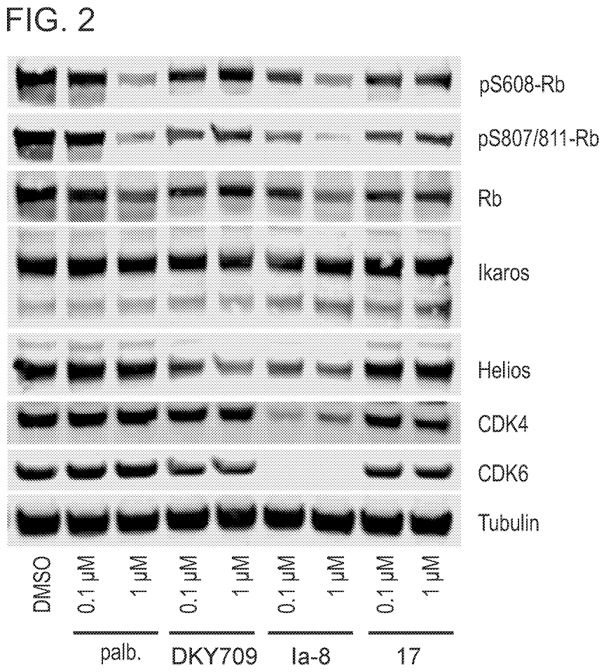

As shown in FIG. 2 (immunoblots from Jurkat cells treated with 1 μM of the indicated compounds for 16 h), palbociclib (CDK4/6 inhibitor) reduced the levels of phosphorylated Rb without affecting the protein abundance of Helios, CDK4, or CDK6, while DKY709 induced Helios degradation without affecting the levels of phosphorylated Rb. Ia-8 induced the triple degradation of Helios, CDK4, and CDK6 and reduced the levels of phosphorylated Rb, while the negative control compound 17 had minimal effects.

### Example 16: Induction of G1 Arrest

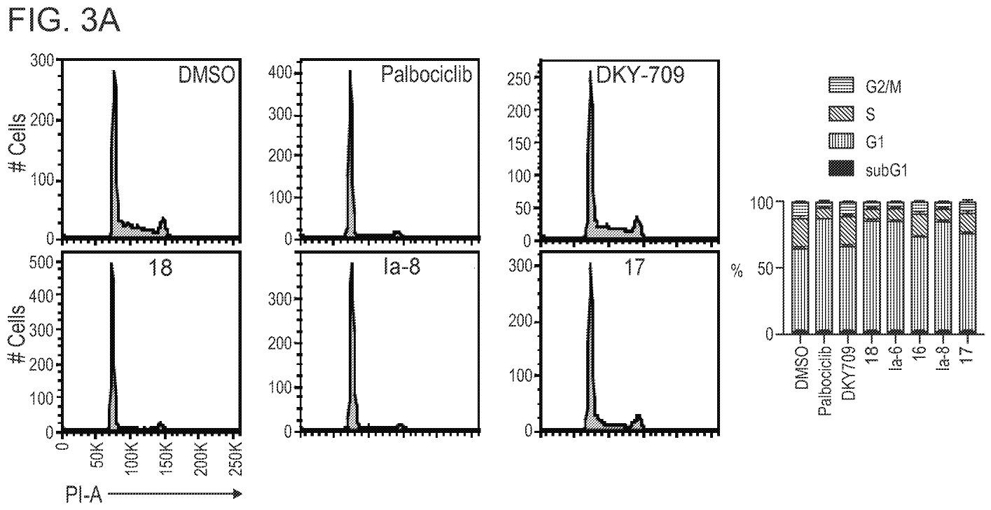

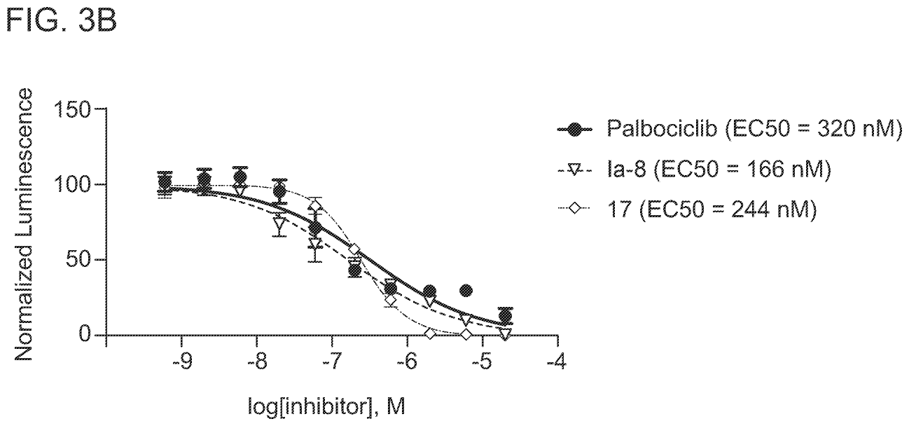

As shown in FIG. 3A (propidium iodide staining for DNA content of Jurkat cells treated with 100 nM of the indicated compound for 24 h), palbociclib, but not DKY709, induced G1 arrest, while 18 and Ia-8 induced G1 arrest, while the negative control compound 17 did not. The structure of 18 (N-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)-2-((2-(1-methyl-2,6-dioxopiperidin-3-yl)-1,3-dioxoisoindolin-4-yl)amino)acetamide) is shown here:

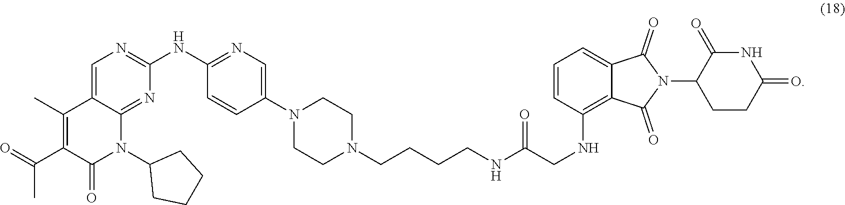 [SMILES: CC(=O)c1c(C)c2cnc(Nc3ccc(N4CCN(CCCCNC(=O)CNc5cccc6c5C(=O)N(C5CCC(=O)NC5=O)C6=O)CC4)cn3)nc2n(C2CCCC2)c1=O]

### Example 17: Measurement of Anti-Proliferative Activity

As shown in FIG. 3B, Jurkat cells were treated with a dose curve of the indicated compounds for 3 d and cell proliferation was quantified by CellTiter-Glo, revealing that the negative control compound 17 had less potent anti-proliferative activity (244 nM) than Ia-8 (166 nM).

### Example 18: CDK4-CDK6-Helios Co-Degradation Enhances IL-2 Secretion

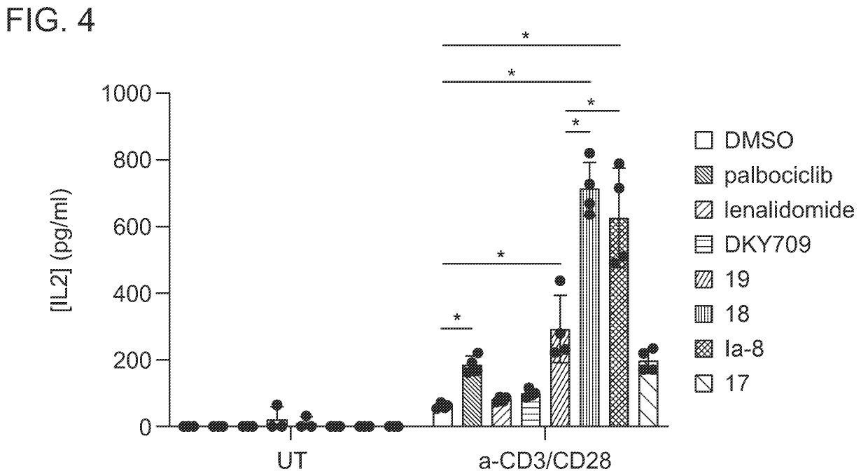

Quantification of IL-2 levels by ELISA from Jurkat cells pre-treated with 1 μM of the indicated compounds for 24 h and then TCR-stimulated for 18 h. Results are shown in FIG. 4 as mean±SD (untreated, n=3; other conditions, n=4) (*p<0.05). Lenalidomide and DKY709 treatment both resulted in elevated IL-2 secretion, although it did not reach statistical significance. While treatment with the CDK4/CDK6-selective degrader, 19, has similar activity as palbociclib, the CDK4/CDK6/Ikaros/Aiolos degrader, 18, induced even greater IL-2 secretion. Notably, treatment with the CDK4/CDK6/Helios triple degrader, Ia-8, induced elevated levels of IL2 that were comparable to that of 18, while the inactive chemical control compound, 17, had activity similar to that of palbociclib. The structure of 19 (N-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)-2-((2-(2,6-dioxopiperidin-3-yl)-1,3-dioxoisoindolin-4-yl)oxy)acetamide) is shown here:

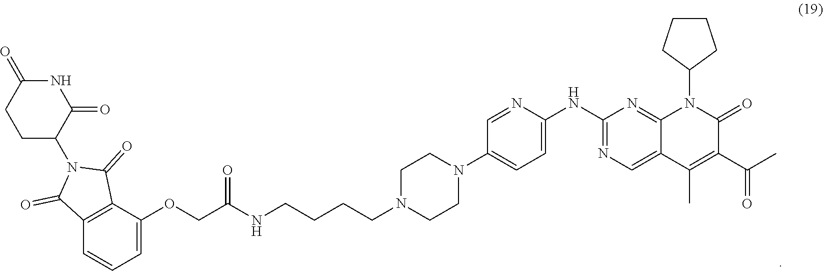 [SMILES: CC(=O)c1c(C)c2cnc(Nc3ccc(N4CCN(CCCCNC(=O)COc5cccc6c5C(=O)N(C5CCC(=O)NC5=O)C6=O)CC4)cn3)nc2n(C2CCCC2)c1=O]

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A bifunctional compound having a structure represented by formula (I): 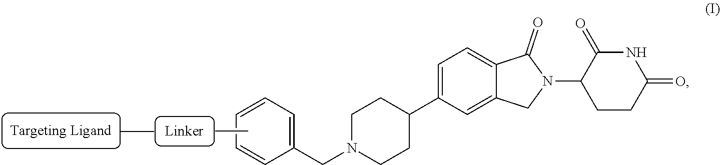
or a pharmaceutically acceptable salt or stereoisomer thereof, wherein the targeting ligand (“Targeting Ligand”) binds CDK4 and/or CDK6, and the linker (“Linker”) comprises an alkylene chain or a polyethylene glycol chain.

2. The bifunctional compound of claim 1 having a structure represented by formula (Ia), formula (Ib), or formula (Ic): 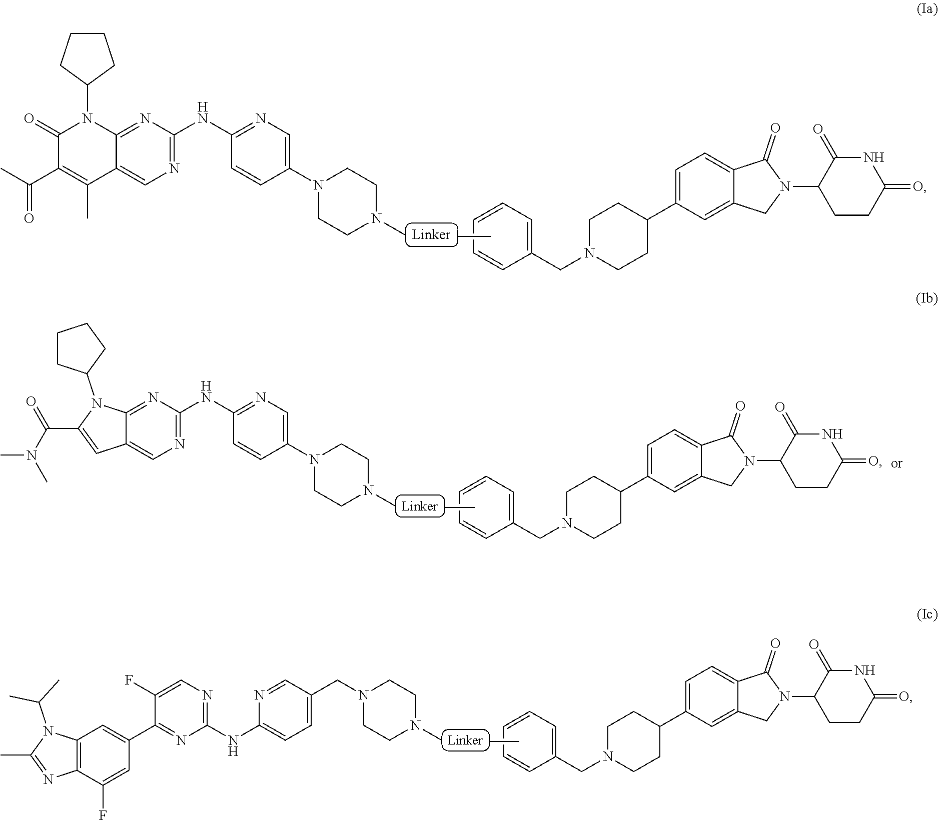
or a pharmaceutically acceptable salt or stereoisomer thereof.

3. The bifunctional compound of claim 1, wherein the linker comprises an alkylene chain.

4. The bifunctional compound of claim 3, wherein the alkylene chain is interrupted by, and/or terminates at either or both termini with, at least one of —O— or —NH—.

5. The bifunctional compound of claim 1, wherein the linker comprises 1 to 6 alkylene units.

6. The bifunctional compound of claim 1, wherein the linker comprises a polyethylene glycol chain.

7. The bifunctional compound of claim 6, wherein the linker terminates at either or both termini with, at least one of —O— or —NH—.

8. The bifunctional compound of claim 6, wherein the linker comprises 2 to 3 ethylene glycol units.

9. The bifunctional compound of claim 1, wherein the Linker is any one of the structures:

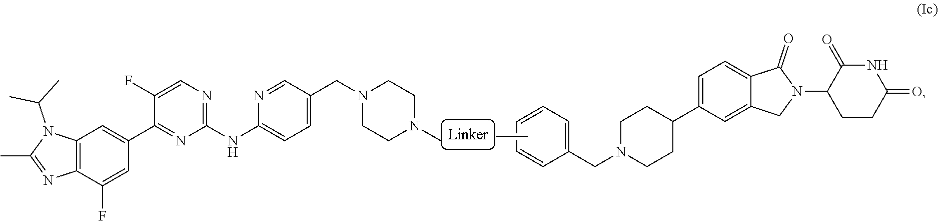

10. The bifunctional compound of claim 1, wherein the Linker is attached to the phenyl group at the 3-position (meta).

11. The bifunctional compound of claim 1, wherein the Linker is attached to the phenyl group at the 4-position (para).

12. The bifunctional compound of claim 1, which is: 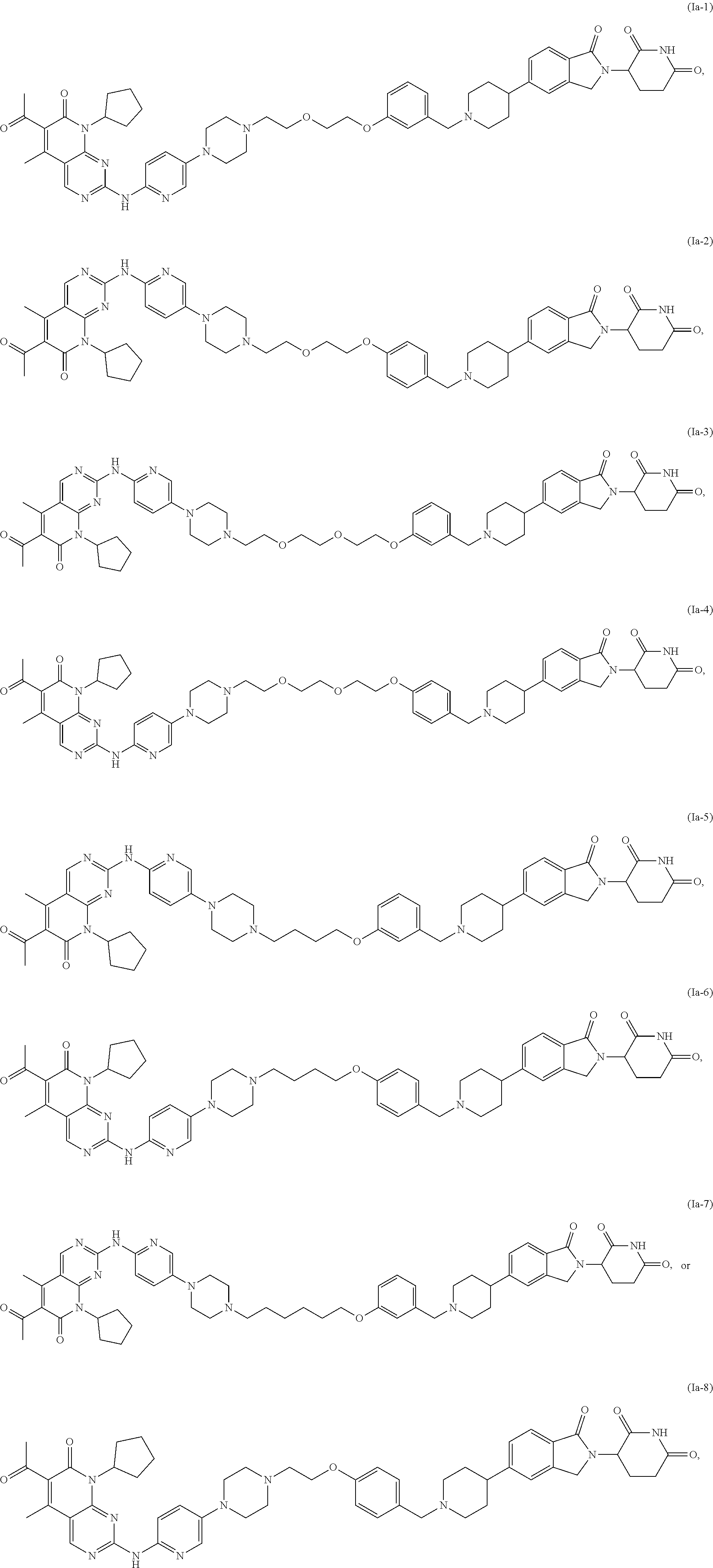
or a pharmaceutically acceptable salt or stereoisomer thereof.

13. A pharmaceutical composition, comprising a therapeutically effective amount of the bifunctional compound or pharmaceutically acceptable salt or stereoisomer thereof of claim 1, and a pharmaceutically acceptable carrier.

14. The pharmaceutical composition of claim 13, which is in the form of a solid.

15. The pharmaceutical composition of claim 14, which is in the form of a tablet or capsule.

16. The pharmaceutical composition of claim 13, which is in the form of a liquid.

17. A method of treating a disease or disorder that is characterized by aberrant activity of CDK4 and/or CDK6, and Helios, comprising administering to a subject in need thereof a therapeutically effective amount of the bifunctional compound or pharmaceutically acceptable salt or stereoisomer thereof of claim 1.

18. The method of claim 17, wherein the disease or disorder is cancer.

19. The method of claim 18, wherein the cancer is characterized by a solid tumor.

20. The method of claim 18, wherein the cancer is selected from breast cancer, brain cancer, endometrial cancer, head and neck cancer, gastrointestinal cancer, lung cancer, ovarian cancer, prostate cancer, uterine cancer, hepatocellular carcinoma, liposarcoma, melanoma, and a hematological cancer.

21. (canceled)

22. The method of claim 20, wherein the hematological cancer is selected from leukemia, lymphoma, and myeloma.

23. A method of reducing the levels of CDK4 and/or CDK6, and Helios in a cell, either in vitro or in vivo, comprising contacting the cell with the bifunctional compound of claim 1.
